(19) United States
(12) Patent Application Publication
(10) Pub. No.: US 2011/0295297 A1
(43) Pub. Date: Dec. 1, 2011

(54) DICE CUTTING SURGICAL INSTRUMENT

(75) Inventors: Brady R. Shirley, Lewisville, TX (US); Daniel E. Gerbec, Logan, UT (US); Joseph Q. Marietta, Hyde Park, UT (US); Brandon T. Walker, Layton, UT (US)

(73) Assignee: MEDICINELODGE, INC. DBA IMDS CO-INNOVATION, Logan, UT (US)

(21) Appl. No.: 13/115,755

(22) Filed: May 25, 2011

Related U.S. Application Data
(60) Provisional application No. 61/348,346, filed on May 26, 2010.

(51) Int. Cl. A61B 17/32

(52) U.S. Cl. 606/184

(57) ABSTRACT

Surgical punch tools remove a waste portion of tissue from a main portion and subdivide the waste portion into two or more smaller debris portions. The tools may also grasp the waste portion or eject the debris from the tool.

### CROSS-REFERENCE TO RELATED APPLICATIONS

This application claims the benefit of the filing date of:

U.S. Provisional Patent Application No. 61/348,346, which was filed on May 26, 2010, is entitled DICE CUTTING SURGICAL INSTRUMENT, and carries Attorney's docket no. IWO-7 PROV. The contents of U.S. Application No. 61/348,346 are hereby incorporated by reference as part of this application.

### BACKGROUND OF THE INVENTION

The present disclosure relates to instruments for tissue removal. While the present disclosure is made in the context of surgical instruments, such as arthroscopic, laparoscopic, endoscopic, minimally invasive instruments, or other instruments, among others, the principles embodied in the present disclosure may be applicable outside the fields of surgery or medical devices.

Tissue removal instruments may be broadly classified according to the way tissue is removed by the instrument. One class of tissue removal instruments may be described as punches. A punch tends to remove a single piece of cut tissue per actuation. The removed piece of tissue may be referred to as a waste portion of the tissue. Punches may also be described as biters. For example, a punch may be designed with opposing jaws to grasp and cut a piece of tissue in a manner similar to a person taking a bite of a sandwich. As a further example, a punch or biter may be designed to remove a generally semicircular, semi-oval, or rectangular piece of tissue per actuation. One can appreciate that a tissue edge that has been treated with such a punch may present a scalloped appearance created by multiple partially overlapping bites along the edge. The rate of tissue removal may be proportional to the bite size of the punch. Thus, a larger bite size may reduce the time needed to remove a given amount of tissue by reducing the number of bites which must be taken.

Once a piece of tissue has been cut by a punch or any other tool, the piece of tissue may act as a piece of debris in the field around the instrument. In arthroscopic surgical procedures, the debris may drift around the joint space before being removed through a suction port or drainage port. Large pieces of debris may be easy to see, but may not pass through the port. Debris that is close to the same size as the port may tend to clog the port rather than passing through it. Small pieces of debris may easily pass through the port, but it may be difficult or time-consuming to verify that all debris has been removed. Therefore, it may be desirable to strike a balance in debris particle size and quantity so that debris is easily removed and its removal is easily verified.

There is a need for punches which take a large bite of tissue and produce two or more smaller pieces of debris per actuation. There is also a need for systems which include such punches and ports through which the smaller pieces of debris readily pass.

### SUMMARY OF THE INVENTION

An embodiment of an instrument includes a first jaw, wherein the first jaw comprises an aperture at least partially encircled by a first cutting edge; a second jaw connected to the first jaw by a hinge, wherein the second jaw rotates relative to the first jaw about an axis of rotation of the hinge; and a second cutting edge, wherein the second cutting edge spans the aperture.

In another embodiment, the second cutting edge extends parallel to the axis.

In yet another embodiment, the second cutting edge extends perpendicular to the axis.

In yet another embodiment, the second cutting edge is carried by the first jaw.

In yet another embodiment, the second cutting edge is carried by the second jaw.

In yet another embodiment, an instrument includes a third cutting edge, wherein the third cutting edge spans the aperture.

In yet another embodiment, the third cutting edge crosses the second cutting edge.

In yet another embodiment, a surgical punch includes a first cutting edge, wherein the first cutting edge extends along a majority of a perimeter around a simple two-dimensional region; and a second cutting edge, wherein the second cutting edge crosses the region, wherein the second cutting edge extends along a path that intersects the first cutting edge.

In yet another embodiment, a punch includes a pair of opposing jaws, wherein the first and second cutting edges are carried by the jaws.

In yet another embodiment, the jaws are connected by a hinge, wherein the hinge extends along a remainder of the perimeter.

In yet another embodiment, the second cutting edge is parallel to the hinge.

In yet another embodiment, the second cutting edge is perpendicular to the hinge.

In yet another embodiment, a punch includes a third cutting edge, wherein the third cutting edge crosses the region, wherein the third cutting edge extends along a path that intersects the first cutting edge.

In yet another embodiment, the third cutting edge extends along a path that intersects the second cutting edge.

In yet another embodiment, a surgical punch includes means for cutting a waste portion of a tissue from a main portion of the tissue; means for subdividing the waste portion into a plurality of debris portions; and means for actuating the punch to cut and subdivide the waste portion; wherein a single actuation of the punch actuates the means for cutting and subdividing the waste portion.

In yet another embodiment, a punch includes a hinged pair of opposing jaws, wherein the means for cutting and subdividing the waste portion are carried on the jaws.

In yet another embodiment, a punch includes means for grasping the waste portion.

In yet another embodiment, a single actuation of a punch actuates the means for grasping, cutting, and subdividing the waste portion.

In yet another embodiment, a punch includes means for ejecting the debris portions.

In yet another embodiment, a single actuation of a punch actuates the means for cutting the waste portion, the means for subdividing the waste portion, and the means for ejecting the debris portions.

In yet another embodiment, in a single actuation, a surgical punch removes a waste portion of the tissue from a main portion of the tissue and divides the waste portion into at least two debris portions.

### BRIEF DESCRIPTION OF THE DRAWINGS

FIG. 1A is a perspective view of a punch; and FIG. 1B is a detail view of a working tip of the punch of FIG. 1A;

FIG. 2 is a broken exploded perspective view of the punch of FIG. 1A;

FIG. 3A is a broken top view of the punch of FIG. 1A with open jaws; FIG. 3B is a broken cross section view of the punch of FIG. 3A taken along line 3B-3B; FIG. 3C is a detail view of a working tip of the punch of FIG. 3B taken at circle 3C; and FIG. 3D is a detail view of a handle portion of the punch of FIG. 3B taken at circle 3D;

FIG. 4A is a broken top view of the punch of FIG. 1A with closed jaws; FIG. 4B is a broken cross section view of the punch of FIG. 4A taken along line 4B-4B; FIG. 4C is a detail view of a working tip of the punch of FIG. 4B taken at circle 4C; and FIG. 4D is a detail view of a handle portion of the punch of FIG. 4B taken at circle 4D;

FIG. 5A is a perspective view of a first jaw of the punch of FIG. 1A; and FIG. 5B is another perspective view of the first jaw of FIG. 5A in a different orientation;

FIG. 6A is a perspective view of a second jaw of the punch of FIG. 1A; and FIG. 6B is another perspective view of the second jaw of FIG. 6A in a different orientation;

FIG. 7A is a perspective view of another punch; and FIG. 7B is a detail view of a working tip of the punch of FIG. 7A;

FIG. 8 is a broken exploded perspective view of the punch of FIG. 7A;

FIG. 9A is a broken top view of the punch of FIG. 7A with open jaws; FIG. 9B is a broken cross section view of the punch of FIG. 9A taken along line 9B-9B; FIG. 9C is a detail view of a working tip of the punch of FIG. 9B taken at circle 9C; and FIG. 9D is a detail view of a handle portion of the punch of FIG. 9B taken at circle 9D;

FIG. 10A is a broken top view of the punch of FIG. 7A with closed jaws; FIG. 10B is a broken cross section view of the punch of FIG. 10A taken along line 10B-10B; FIG. 10C is a detail view of a working tip of the punch of FIG. 10B taken at circle 10C; and FIG. 10D is a detail view of a handle portion of the punch of FIG. 10B taken at circle 10D;

FIG. 11A is a perspective view of a first jaw of the punch of FIG. 7A; and FIG. 11B is another perspective view of the first jaw of FIG. 11A in a different orientation;

FIG. 12A is a perspective view of a second jaw of the punch of FIG. 7A; and FIG. 12B is another perspective view of the second jaw of FIG. 12A in a different orientation;

FIG. 13 is a detail view of a working tip of yet another punch;

FIG. 14A is a perspective view of yet another punch; and FIG. 14B is a detail view of a working tip of the punch of FIG. 14A;

FIG. 15 is a broken exploded perspective view of the punch of FIG. 14A;

FIG. 16A is a broken top view of the punch of FIG. 14A with open jaws; FIG. 16B is a broken cross section view of the punch of FIG. 16A taken along line 16B-16B; FIG. 16C is a detail view of a working tip of the punch of FIG. 16B taken at circle 16C; and FIG. 16D is a detail view of a handle portion of the punch of FIG. 16B taken at circle 16D;

FIG. 17A is a broken top view of the punch of FIG. 14A with closed jaws; FIG. 17B is a broken cross section view of the punch of FIG. 17A taken along line 17B-17B; FIG. 17C is a detail view of a working tip of the punch of FIG. 17B taken at circle 17C; and FIG. 17D is a detail view of a handle portion of the punch of FIG. 17B taken at circle 17D;

FIG. 18A is a perspective view of a first jaw of the punch of FIG. 14A; and FIG. 18B is another perspective view of the first jaw of FIG. 18A in a different orientation;

FIG. 19A is a perspective view of a second jaw of the punch of FIG. 14A; and FIG. 19B is another perspective view of the second jaw of FIG. 19A in a different orientation;

FIG. 20A is a perspective view of yet another punch; and FIG. 20B is a detail view of a working tip of the punch of FIG. 20A;

FIG. 21 is a broken exploded perspective view of the punch of FIG. 20A;

FIG. 22A is a broken top view of the punch of FIG. 20A with open jaws; FIG. 22B is a broken cross section view of the punch of FIG. 22A taken along line 22B-22B; FIG. 22C is a detail view of a working tip of the punch of FIG. 22B taken at circle 22C; and FIG. 22D is a detail view of a handle portion of the punch of FIG. 22B taken at circle 22D;

FIG. 23A is a broken top view of the punch of FIG. 20A with closed jaws; FIG. 23B is a broken cross section view of the punch of FIG. 23A taken along line 23B-23B; FIG. 23C is a detail view of a working tip of the punch of FIG. 23B taken at circle 23C; and FIG. 23D is a detail view of a handle portion of the punch of FIG. 23B taken at circle 23D;

FIG. 24A is a perspective view of a first jaw of the punch of FIG. 20A; and FIG. 24B is another perspective view of the first jaw of FIG. 24A in a different orientation;

FIG. 25A is a perspective view of a second jaw of the punch of FIG. 20A; and FIG. 25B is another perspective view of the second jaw of FIG. 25A in a different orientation; and

FIG. 26 is a detail view of a working tip of yet another punch.

### DETAILED DESCRIPTION

While certain embodiments have been shown and described in detail below by way of illustration only, it will be clear to the person skilled in the art upon reading and understanding this disclosure that changes, modifications, and variations may be made and remain within the scope of the systems, kits, components, and methods described herein. Furthermore, while various features are grouped together in the embodiments for the purpose of streamlining the disclosure, it is appreciated that features from different embodiments may be combined to form additional embodiments which are all contemplated within the scope of the present disclosure.

Not every feature of each embodiment is labeled in every figure in which that embodiment appears, in order to keep the figures clear. Similar reference numbers (for example, those that are identical except for the first numeral) are used to indicate similar features in different embodiments.

Standard medical planes of reference and descriptive terminology are employed in this specification. A sagittal plane divides a body into right and left portions. A mid-sagittal plane divides the body into equal right and left halves. A coronal plane divides a body into anterior and posterior portions. A transverse plane divides a body into superior and inferior portions. Anterior means toward the front of the body. Posterior means toward the back of the body. Superior means toward the head. Inferior means toward the feet. Medial means toward the midline of the body. Lateral means away from the midline of the body. Axial means toward a central axis of the body. Abaxial means away from a central axis of the body. These descriptive terms may be applied to an animate or inanimate body. Ipsilateral means on the same side of the body. Contralateral means on the opposite side of the body.

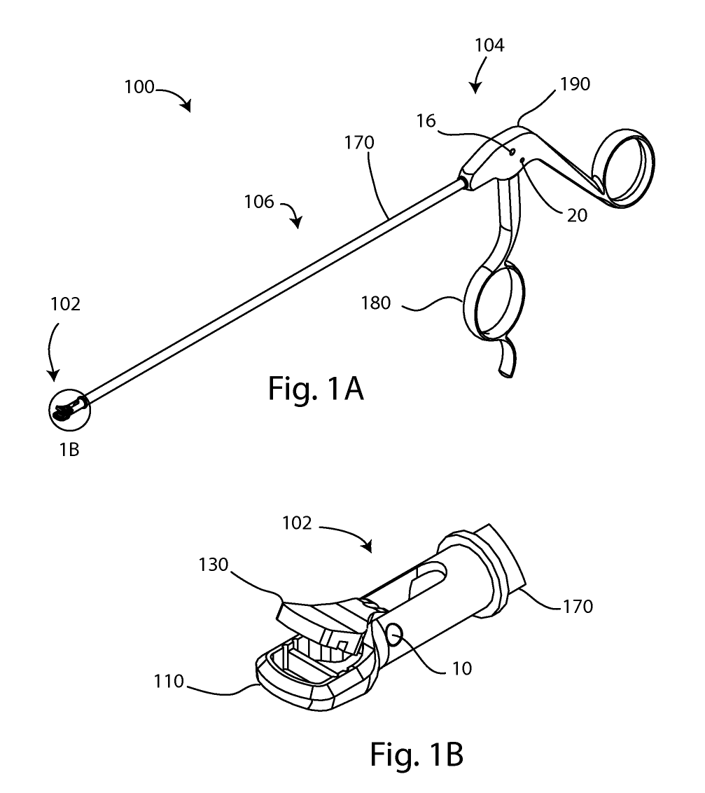

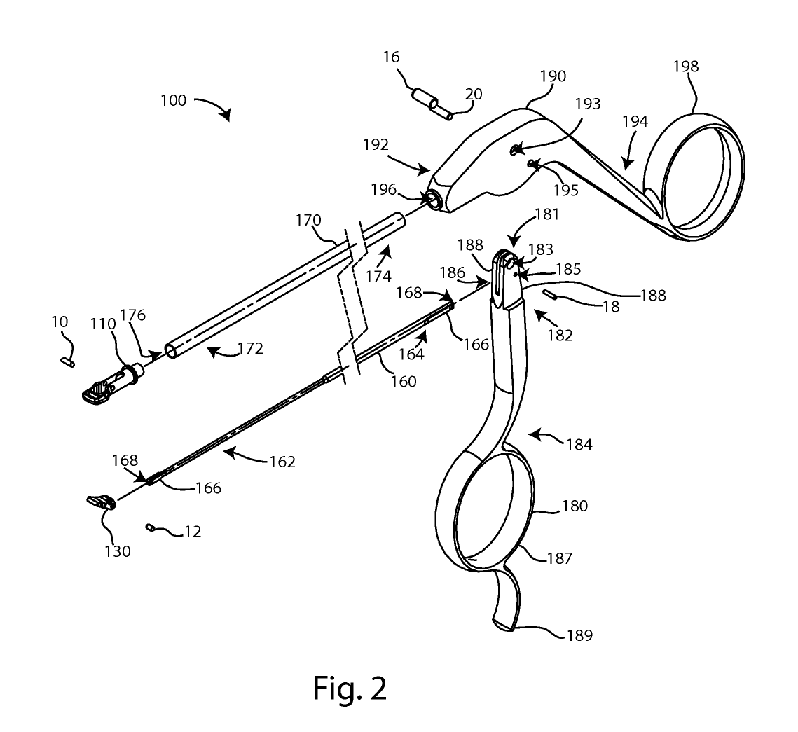

In FIGS. 1A-6B, a punch 100 is illustrated. Punch 100 may include first jaw 110, second jaw 130, first shaft 160, second shaft 170, first handle 180, and second handle 190. Punch 100 may be described as having a working tip 102 and a handle portion 104 separated by a shaft portion 106. The working tip 102 includes means for cutting a single bite of tissue and subdividing the bite into multiple pieces. The single bite of tissue may be described as a waste portion of the tissue. The multiple pieces may be described as debris portions of the tissue. Working tip 102 may include the first and second jaws 110, 130, and may also include an adjacent portion of the first and/or second shafts 160, 170. The handle portion 104 includes means for actuating the punch 100 to cut and subdivide the bite of tissue. Handle portion 104 may include the first and second handles 180, 190, and may also include an adjacent portion of the first and/or second shafts 160, 170. The shaft portion 106 includes at least a middle portion of the first and second shafts 160, 170.

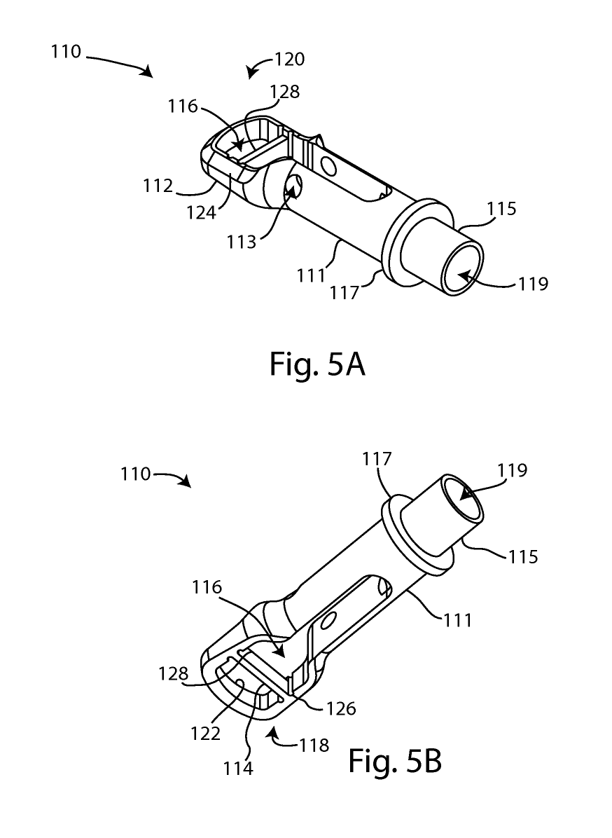

With primary reference to FIGS. 5A-5B, the first jaw 110 includes a frame, rim, or loop 112 and a crossbar 114. The loop 112 encircles a port, or aperture 116 which extends through the first jaw 110 between an outer side 118 and an inner side 120 of the first jaw 110. The aperture 116 may be described as piercing the first jaw 110. The aperture 116 may have a profile that is oblong, rectangular, polygonal, oval, round, or otherwise forms a boundary or perimeter of a simple 2-dimensional region, otherwise known as a closed shape. For example, the aperture 116 illustrated in FIGS. 5A-5B may be described as spoon-shaped or pear-shaped. A cutting edge 122 may extend at least partially around the loop 112 on the inner side 120. For example, the cutting edge 122 may be the edge at the intersection of the aperture 116 and the inner side 120 of the first jaw 110, as illustrated in FIGS. 5A-5B. The cutting edge 122 may be described as following a majority, or at least a portion, of the profile of the aperture 116. The first jaw 110 may include one or more bevels 124. For example, bevel 124 may exist on the inner side 120 of the loop 112, and may be complementary to the cutting edge 122. The loop 112 may include one or more grooves 126 which extend between the outer and inner sides 118, 120 and interrupt the cutting edge 122. The crossbar 114 extends transversely across the loop 112 so as to span, or bridge, the aperture 116. The crossbar 114 may be said to follow a path that intersects the loop 112. The crossbar 114 may be flush with the outer side 118 and recessed below the cutting edge 122. The crossbar 114 includes at least one cutting edge 128 on the inner side 120; FIGS. 5A-5B illustrate a crossbar with two cutting edges. The first jaw 110 may also include a shaft 111 extending from the loop 112. For example, FIGS. 5A-5B illustrate shaft 111 extending from the loop 112 at a narrow end of the spoon-shaped or pear-shaped aperture 116. The shaft 111 may include a transverse through hole 113, which in the illustrated example is on the shaft adjacent to a transition between the narrow end and a wide end of the spoon-shaped or pear-shaped aperture 116. The transverse hole 113 is parallel to the crossbar 114 and intersects the aperture 116. The shaft 111 may terminate in a boss 115, which may be of a different diameter than the shaft 111. A flange 117 may separate the shaft 111 and the boss 115. The shaft 111 may include a central longitudinal hole 119 which extends between the aperture 116 and the boss 115.

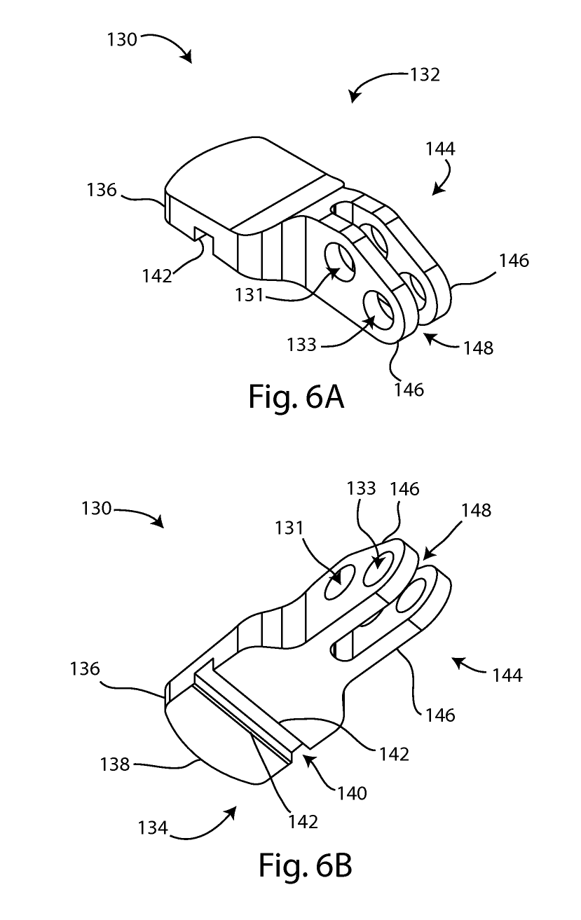

With primary reference to FIGS. 6A-6B, the second jaw 130 may have an outer side 132 and an inner side 134 with a side wall 136 extending around the second jaw 130 between the outer and inner sides 132, 134. The side wall 136 may form a profile which is complementary to at least a portion of the aperture 116 of the first jaw 110. In other words, the side wall 136 may fit the aperture 116, for example with a clearance fit or an interference fit. Referring to FIGS. 1B and 5A-6B, one can appreciate that second jaw 130 and aperture 116 have complementary spoon-shaped or pear-shaped profiles. The second jaw 130 includes a cutting edge 138 on the inner side 134, which may be, for example, the edge at the intersection of the side wall 136 and the inner side 134. The side wall 136 may intersect the inner side 134 at an acute angle in order to sharpen the cutting edge 138. For example, the side wall may be drafted, relieved, or ground to sharpen the cutting edge 138. The second jaw 130 includes a recess, slot, channel, or groove 140 which extends transversely across the second jaw 130. The groove 140 includes at least one cutting edge 142 on the inner side 134; FIGS. 6A-6B illustrate a groove with two cutting edges. The second jaw 130 may form a fork or clevis 144 extending from a narrow end of the spoon-shaped or pear-shaped profile. The clevis 144 may be described as a plurality of arms 146 separated by at least one slot 148. The clevis 144 may include a transverse through hole 131, which in the illustrated example is proximate the outer side 132 adjacent to a transition between the narrow end and a wide end of the spoon-shaped or pear-shaped profile. Hole 131 is parallel to the groove 140. The clevis 144 may also include another transverse through hole 133, which in the illustrated example is proximate the inner side 134 adjacent free ends of the arms 146.

The second jaw 130 may be hinged to the first jaw 110 with fastener 10 in holes 113, 131 so that the inner sides 120, 134 are facing, the second jaw 130 fits the aperture 116, and the groove 140 receives the crossbar 114. One can appreciate that the second jaw 130 may rotate relative to the first jaw 110 about an axis of rotation of fastener 10, which may be a screw or pin. For example, second jaw 130 may rotate relative to first jaw 110 about a central longitudinal axis of fastener 10. It can be appreciated that the hinged connection between the first and second jaws 110, 130 may serve to close off a reduced portion of the aperture 116. In this sense, the hinged connection may be considered to form a portion of a perimeter of the reduced portion of the aperture 116.

Referring to FIGS. 1-4D, the first shaft 160 is an elongated member that extends between a first end portion 162 and a second end portion 164. Each end portion 162, 164 may be flattened so as to form a tang 166, and each end portion may include a through hole 168. First shaft 160 may be rigid or flexible, solid or segmented. For example, first shaft 160 may be a multi-stranded cable.

The first shaft 160 may be hinged to the second jaw 130 with fastener 12 in holes 133, 168 so that the tang 166 on the first end portion 162 fits in the slot 148 of the second jaw.

The second shaft 170 is an elongated member that extends between a first end portion 172 and a second end portion 174. The illustrated shaft 170 is a tube with a central longitudinal through hole 176. The first end portion 172 may include a socket 178 which may be of a different diameter than the hole 176. For example, the socket 178 may be a counterbore.

The second shaft 170 may slidingly receive the first shaft 160 within the hole 176, and may receive the boss 115 of the first jaw 110 within the socket 178. The second shaft 170 and the first jaw 110 may be permanently connected, such as by welding, soldering, brazing, or gluing, among other processes. The flange 117 may provide filler material for a thermal fastening operation. The second shaft 170 and the first jaw 110 may be releasably connected, such as by threads, snap fit, retaining ring, or interference fit, among other connections.

The first handle 180 is an elongated member that extends between a first end portion 182 and a second end portion 184. The first handle 180 may be described as a movable handle. The first end portion 182 may terminate in a bifurcation or clevis 186. The clevis 186 may be described as a plurality of arms 188 separated by at least one slot 181. The clevis 186 may include a transverse through hole 183, which in the illustrated example is proximate terminal ends of the arms 188. The clevis 186 may also include another transverse through hole 185, which in the illustrated example is at an intermediate location along the arms 188. The second end portion 184 may include a loop 187 and/or a rest 189. A user may insert one or more digits through the loop 187 or may rest one or more digits on the rest 189.

The first handle 180 may be hinged to the first shaft 160 with fastener 18 in holes 185, 168 so that the tang 166 on the second end portion 164 fits in the slot 181 of the first handle.

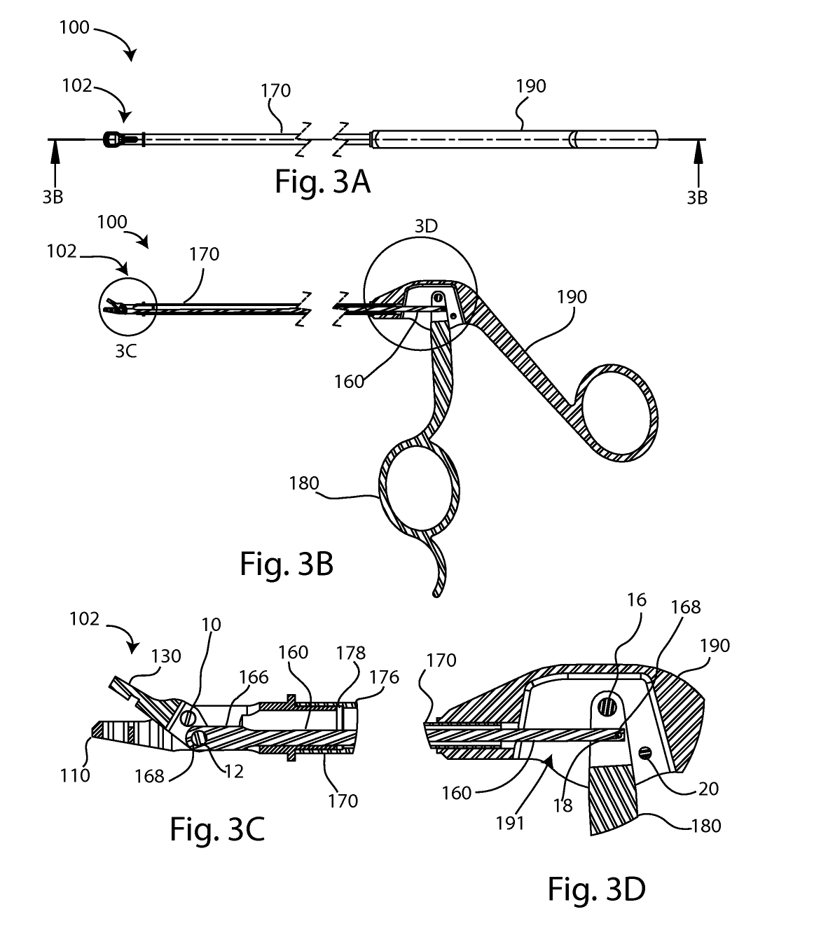

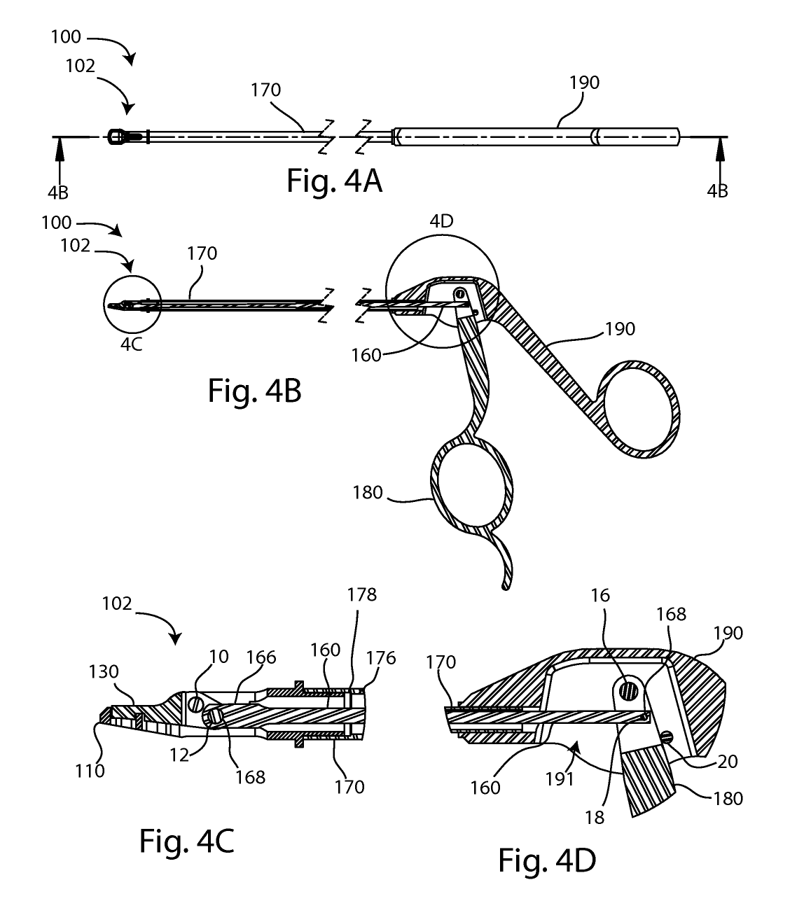

The second handle 190 is an elongated member that extends between a first end portion 192 and a second end portion 194. The second handle 190 may be described as a stationary handle. The first end portion 192 may terminate in a central longitudinal hole 196. The second end portion 194 may include a loop 198, which may resemble loop 187 in appearance and/or function. The second handle 190 may include an indentation, recess, or alcove 191 at an intermediate location between the first and second end portions 192, 194, as may be seen best in FIGS. 3D and 4D. The second handle 190 may include a transverse through hole 193 which intersects, or crosses, the alcove 191. The second handle 190 may include another transverse through hole 195 which also crosses the alcove 191. The holes 193, 195 may be on opposite sides of the hole 196, as shown.

The second handle 190 may be hinged to the first handle 180 with fastener 16 in holes 193, 183 so that alcove 191 receives clevis 186 and hole 185 is proximate hole 195 (FIGS. 3D and 4D). Fastener 20 may be secured in hole 195 to limit the rotation of first handle 180 relative to second handle 190. Hole 196 may receive the second end portion 174 of second shaft 170. The second handle 190 and the second shaft 170 may be permanently or releasably connected in a manner similar to that set forth above with regard to second shaft 170 and first jaw 110. If needed, sacrificial filler material may be provided on the second handle 190 or second shaft 170.

When punch 100 is operatively assembled, the second handle 190, second shaft 170, and first jaw 110 are firmly fixed together and the first handle 180, first shaft 160, and second jaw 130 are linked together so that rotation of first handle 180 relative to second handle 190 about fastener 16 causes corresponding rotation of second jaw 130 relative to first jaw 110 about fastener 10. The illustrated example of punch 100 is configured so that when first handle 180 abuts fastener 20, second jaw 130 fits at least partially within aperture 116 of first jaw 110, as may be seen best in FIGS. 4A-4D. In other words, FIGS. 4A-4D show punch 100 in a configuration in which the jaws 110, 130 are closed. When first handle 180 is rotated away from fastener 20, second jaw 130 tilts away from first jaw 110, as may be seen best in FIGS. 3A-3D. In other words, FIGS. 3A-3D show punch 100 in a configuration in which the jaws 110, 130 are open. It can be appreciated that, as punch 100 is actuated to move from the open configuration to the closed configuration, complementary cutting edges 122 and 138 bypass each other in a shearing action. Similarly, complementary cutting edges 128, 142 bypass each other in a shearing action. As punch 100 moves from the open configuration to the closed configuration with a piece of tissue interposed between the jaws, the tissue will be grasped between the jaws 110, 130 and cut or sheared between edges 128, 138 and between edges 128, 142. A single actuation of the punch 100 takes a large bite of tissue and produces two smaller pieces of tissue debris. Furthermore, as the jaws 110, 130 close, the jaw 130 pushes the smaller pieces of tissue through the aperture 116 to eject the tissue from the punch 100. The aperture 116 may be flared or drafted to be larger on the outer side 118 to assist in ejecting debris from the punch 100. The jaws 110, 130 may close more than is strictly necessary for tissue cutting, in order to push debris through the aperture. The inner side 134 of jaw 130 may include features, such as teeth or posts, which urge debris out of the working tip 102. Therefore, the jaw 130 may be considered a means for ejecting the debris from the punch.

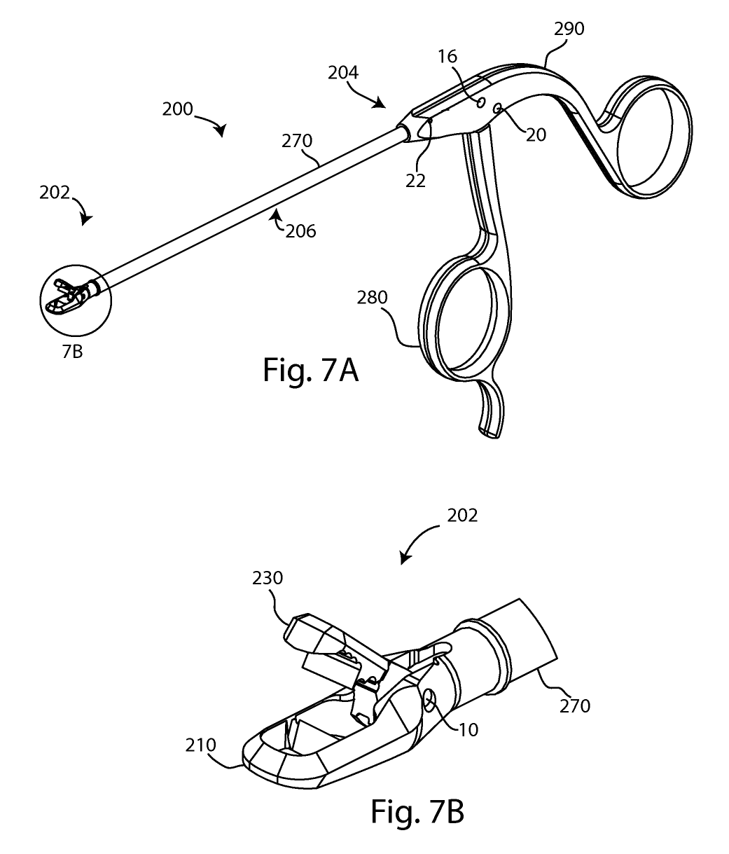

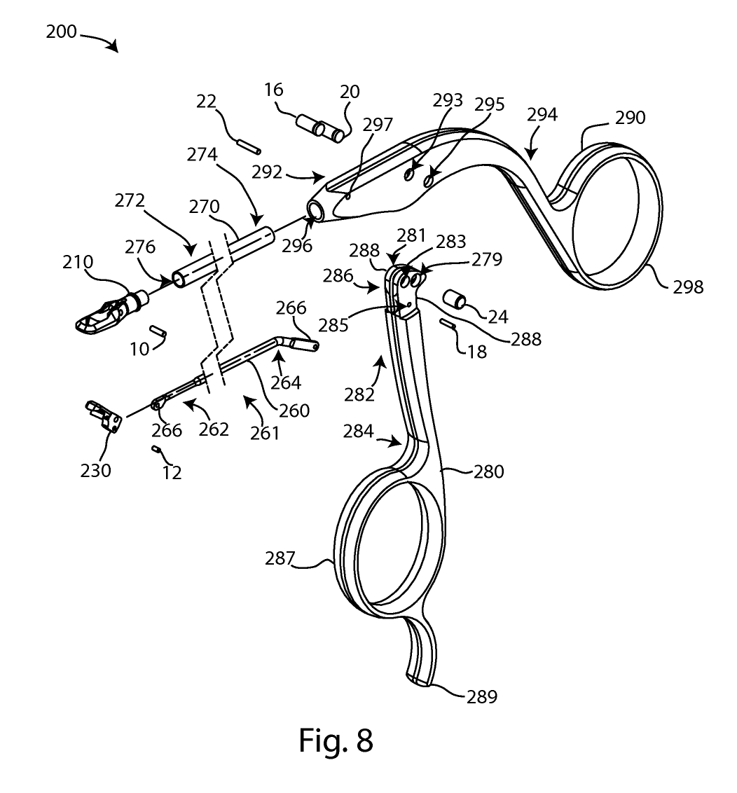

In FIGS. 7A-12B, another punch 200 is illustrated. Punch 200 may include first jaw 210, second jaw 230, first shaft 260, second shaft 270, first handle 280, and second handle 290. Punch 200 may be described as having a working tip 202 and a handle portion 204 separated by a shaft portion 206. The working tip 202 includes means for cutting a single bite of tissue and subdividing the bite into multiple pieces. The single bite of tissue may be described as a waste portion of the tissue. The multiple pieces may be described as debris portions of the tissue. Working tip 202 may include the first and second jaws 210, 230, and may also include an adjacent portion of the first and/or second shafts 260, 270. The handle portion 204 includes means for actuating the punch 200 to cut and subdivide the bite of tissue. Handle portion 204 may include the first and second handles 280, 290, and may also include an adjacent portion of the first and/or second shafts 260, 270. The shaft portion 206 includes at least a middle portion of the first and second shafts 260, 270.

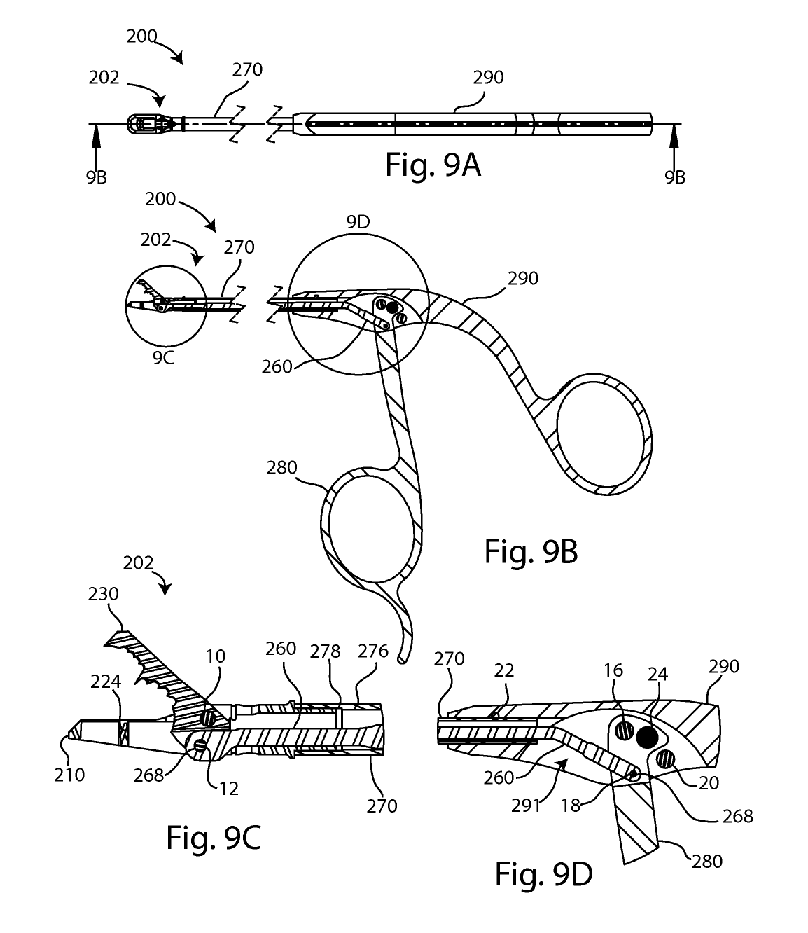

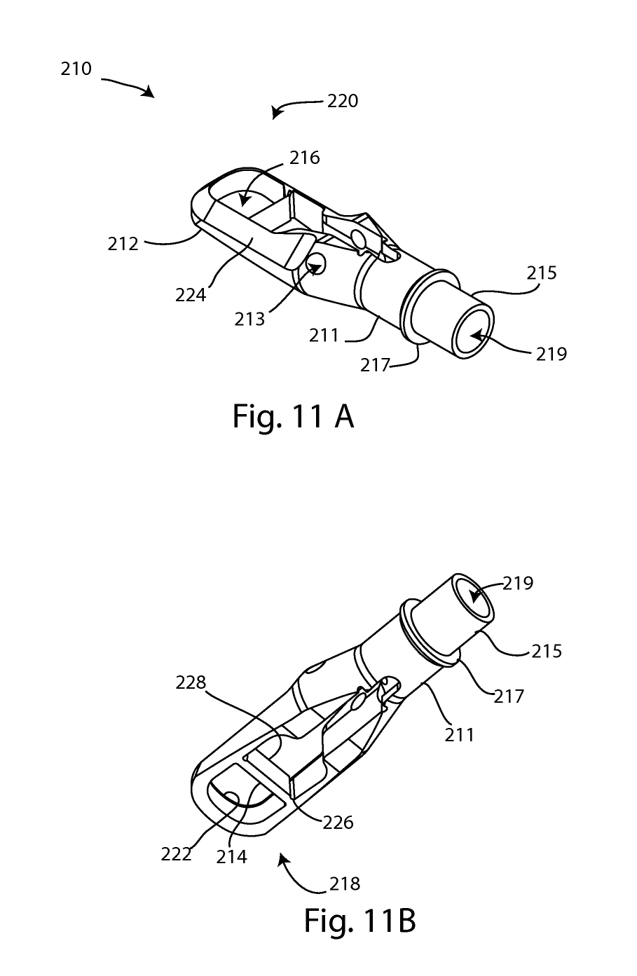

With primary reference to FIGS. 11A-11B, the first jaw 210 includes a frame, rim, or loop 212 and a crossbar 214. The loop 212 encircles an aperture 216 which extends through the first jaw 210 between an outer side 218 and an inner side 220 of the first jaw 210. The aperture 216 may be described as piercing the first jaw 210. The aperture 216 may have a profile that is oblong, rectangular, polygonal, oval, round, or otherwise forms a boundary or perimeter of a simple 2-dimensional region, otherwise known as a closed shape. For example, the aperture 216 illustrated in FIGS. 11A-11B may be described as spoon-shaped or pear-shaped. A cutting edge 222 may extend at least partially around the loop 212 on the inner side 220. For example, the cutting edge 222 may be the edge at the intersection of the aperture 216 and the inner side 220 of the first jaw 210, as illustrated in FIGS. 11A-11B. The cutting edge 222 may be described as following a majority, or at least a portion, of the profile of the aperture 216. The first jaw 210 may include one or more bevels 224. For example, bevel 224 may exist on the inner side 220 of the loop 212, and may be complementary to the cutting edge 222. The loop 212 may include one or more grooves 226 which extend between the outer and inner sides 218, 220 and interrupt the cutting edge 222. The crossbar 214 extends transversely across the loop 212 so as to span, or bridge, the aperture 216. The crossbar 214 may be said to follow a path that intersects the loop 212. The crossbar 214 may be flush with the outer side 218 and recessed below the cutting edge 222. The crossbar 214 includes at least one cutting edge 228 on the inner side 220; FIGS. 11A-11B illustrate a crossbar with one cutting edge and a bevel 224. The bevel 224 may be seen best in FIG. 9C. The first jaw 210 may also include a shaft 211 extending from the loop 212. For example, FIGS. 11A-11B illustrate shaft 211 extending from the loop 212 at a narrow end of the spoon-shaped or pear-shaped aperture 216. The shaft 211 may include a transverse through hole 213, which in the illustrated example is on the shaft in the narrow end of the spoon-shaped or pear-shaped aperture 216. The transverse hole 213 is parallel to the crossbar 214 and intersects the aperture 216. The shaft 211 may terminate in a boss 215, which may be of a different diameter than the shaft 211. A flange 217 may separate the shaft 211 and the boss 215. The shaft 211 may include a central longitudinal hole 219 which extends between the aperture 216 and the boss 215.

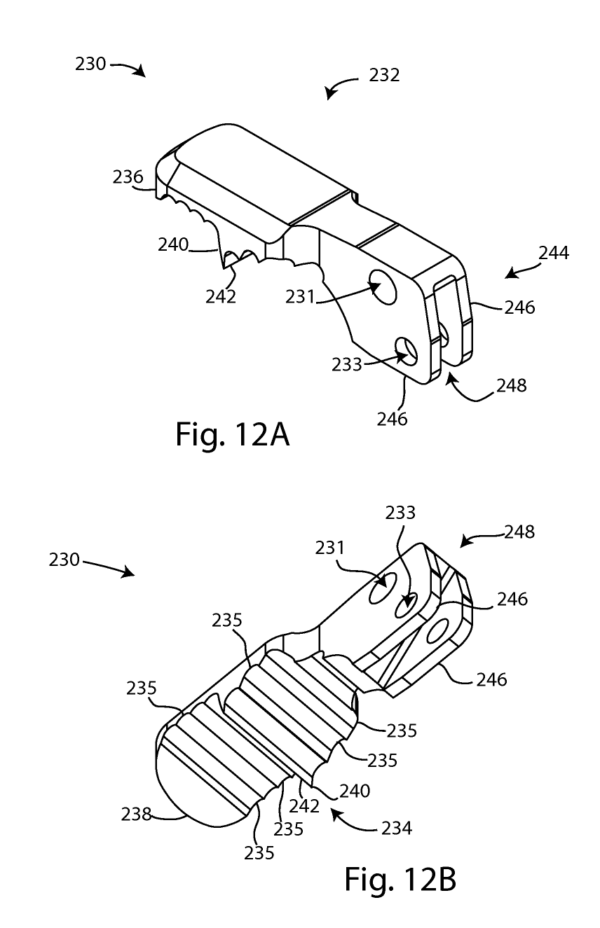

With primary reference to FIGS. 12A-12B, the second jaw 230 may have an outer side 232 and an inner side 234 with a side wall 236 extending around the second jaw 230 between the outer and inner sides 232, 234. The side wall 236 may form a profile which is complementary to at least a portion of the aperture 216 of the first jaw 210. In other words, the side wall 236 may fit the aperture 216, for example with a clearance fit or an interference fit. Referring to FIGS. 7B and 11A-12B, one can appreciate that second jaw 230 and aperture 216 have complementary spoon-shaped or pear-shaped profiles. The second jaw 230 includes a cutting edge 238 on the inner side 234, which may be, for example, the edge at the intersection of the side wall 236 and the inner side 234. The side wall 236 may intersect the inner side 234 at an acute angle in order to sharpen the cutting edge 238. For example, the side wall may be drafted, relieved, or ground to sharpen the cutting edge 238. The second jaw 230 may include one or more grooves 235 which interrupt, or senate, the cutting edge 238. The grooves 235 also produce a texture on the inner side 234 of the second jaw, which may aid in grasping tissue prior to cutting the tissue. Thus, the grooves may be an example of a means for grasping tissue. Each groove 235 may penetrate into the second jaw 230 to a different depth so that a desired tissue grasping effect is achieved at various positions in the actuation cycle. The second jaw 230 includes a crossbar 240 which extends transversely across the second jaw 230. The crossbar 240 includes at least one cutting edge 242 on the inner side 234; FIGS. 12A-12B illustrate a crossbar with one cutting edge. The second jaw 230 may form a fork or clevis 244 extending from a narrow end of the spoon-shaped or pear-shaped profile. The clevis 244 may be described as a plurality of arms 246 separated by at least one slot 248. The clevis 244 may include a transverse through hole 231, which in the illustrated example is proximate the outer side 232 in the narrow end of the spoon-shaped or pear-shaped profile. Hole 231 is parallel to the crossbar 240. The clevis 244 may also include another transverse through hole 233, which in the illustrated example is proximate the inner side 234 adjacent free ends of the arms 246.

The second jaw 230 may be hinged to the first jaw 210 with fastener 10 in holes 213, 231 so that the inner sides 220, 234 are facing, the second jaw 230 fits the aperture 216, and the crossbar 240 engages the crossbar 214. One can appreciate that the second jaw 230 may rotate relative to the first jaw 210 about an axis of rotation of fastener 10, which may be a screw or pin. For example, second jaw 230 may rotate relative to first jaw 210 about a central longitudinal axis of fastener 10. It can be appreciated that the hinged connection between the first and second jaws 210, 230 may serve to close off a reduced portion of the aperture 216. In this sense, the hinged connection may be considered to form a portion of a perimeter of the reduced portion of the aperture 216.

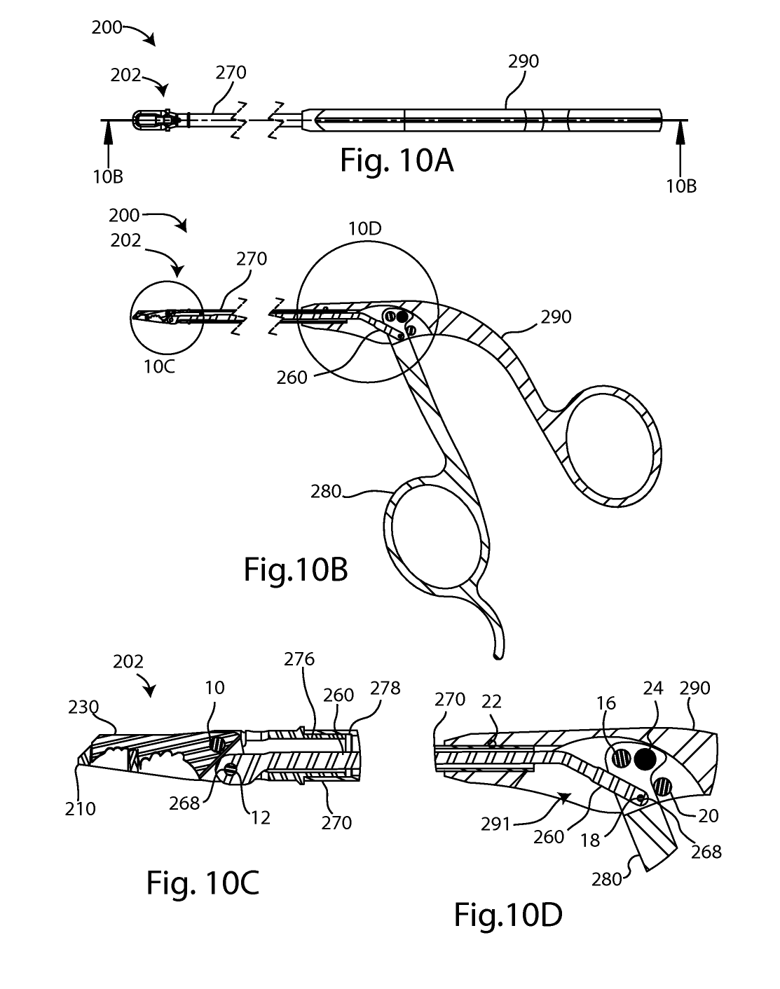

Referring to FIGS. 7A-10D, the first shaft 260 is an elongated member that extends between a first end portion 262 and a second end portion 264. Each end portion 262, 264 may be flattened so as to form a tang 266, and each end portion may include a through hole 268. Each end portion 262, 264 may be positioned eccentrically relative to a mid portion 261 of the first shaft 260. For example, the tang 266 of first end portion 262 is eccentrically laterally enlarged (FIGS. 9C and 10C), while second end portion 264 is bent (FIGS. 9D and 10D). First shaft 260 may be rigid or flexible, solid or segmented. For example, first shaft 260 may be a multi-stranded cable, and may include attached end fittings which form the tangs 266.

The first shaft 260 may be hinged to the second jaw 230 with fastener 12 in holes 233, 268 so that the tang 266 on the first end portion 262 fits in the slot 248 of the second jaw.

The second shaft 270 is an elongated member that extends between a first end portion 272 and a second end portion 274. The illustrated shaft 270 is a tube with a central longitudinal through hole 276. The first end portion 272 may include a socket 278 which may be of a different diameter than the hole 276. For example, the socket 278 may be a counterbore.

The second shaft 270 may slidingly receive the first shaft 260 within the hole 276, and may receive the boss 215 of the first jaw 210 within the socket 278. The second shaft 270 and the first jaw 210 may be permanently connected, such as by welding, soldering, brazing, or gluing, among other processes. The flange 217 may provide filler material for a thermal fastening operation. The second shaft 270 and the first jaw 210 may be releasably connected, such as by threads, snap fit, retaining ring, or interference fit, among other connections.

The first handle 280 is an elongated member that extends between a first end portion 282 and a second end portion 284. The first handle 280 may be described as a movable handle. The first end portion 282 may terminate in a bifurcation or clevis 286. The clevis 286 may be described as a plurality of arms 288 separated by at least one slot 281. The clevis 286 may include a transverse through hole 283, which in the illustrated example is proximate terminal ends of the arms 288. The clevis 286 may also include another transverse through hole 285, which in the illustrated example is at an intermediate location along the arms 288. The clevis 286 may include yet another transverse through hole 279 next to hole 283. The second end portion 284 may include a loop 287 and/or a rest 289. A user may insert one or more digits through the loop 287 or may rest one or more digits on the rest 289.

The first handle 280 may be hinged to the first shaft 260 with fastener 18 in holes 285, 268 so that the tang 266 on the second end portion 264 fits in the slot 281 of the first handle. Fastener 24 may be secured in hole 279. Fastener 24 may, for example, function as an attachment point for a spring (not shown) to bias the first handle 280 when the punch 200 is fully assembled. Alternatively, fastener 24 may function as a drag plug by sliding with friction in the complete assembly.

The second handle 290 is an elongated member that extends between a first end portion 292 and a second end portion 294. The second handle 290 may be described as a stationary handle. The first end portion 292 may terminate in a central longitudinal hole 296. A transverse through hole 297 may extend through the first end portion 292 more or less tangent to the hole 296. The second end portion 294 may include a loop 298, which may resemble loop 287 in appearance and/or function. The second handle 290 may include an indentation, recess, or alcove 291 at an intermediate location between the first and second end portions 292, 294, as may be seen best in FIGS. 9D and 10D. The second handle 290 may include a transverse through hole 293 which intersects, or crosses, the alcove 291. The second handle 290 may include another transverse through hole 295 which also crosses the alcove 291. The holes 293, 295 may be on opposite sides of the hole 296, as shown.

The second handle 290 may be hinged to the first handle 280 with fastener 16 in holes 293, 283 so that alcove 291 receives clevis 286 and hole 285 is proximate hole 295 (FIGS. 9D and 10D). Fastener 20 may be secured in hole 295 to limit the rotation of first handle 280 relative to second handle 290. Hole 296 may receive the second end portion 274 of second shaft 270. The second handle 290 and the second shaft 270 may be permanently or releasably connected in a manner similar to that set forth above with regard to second shaft 270 and first jaw 210. If needed, sacrificial filler material may be provided on the second handle 290 or second shaft 270. In FIGS. 9D and 10D, the second handle 290 and the second shaft 270 are connected by inserting fastener 22 in hole 297, for example by force fitting.

When punch 200 is operatively assembled, the second handle 290, second shaft 270, and first jaw 210 are firmly fixed together and the first handle 280, first shaft 260, and second jaw 230 are linked together so that rotation of first handle 280 relative to second handle 290 about fastener 16 causes corresponding rotation of second jaw 230 relative to first jaw 210 about fastener 10. The illustrated example of punch 200 is configured so that when first handle 280 abuts fastener 20, second jaw 230 fits at least partially within aperture 216 of first jaw 210, as may be seen best in FIGS. 10A-10D. In other words, FIGS. 10A-10D show punch 200 in a configuration in which the jaws 210, 230 are closed. When first handle 280 is rotated away from fastener 20, second jaw 230 tilts away from first jaw 210, as may be seen best in FIGS. 9A-9D. In other words, FIGS. 9A-9D show punch 200 in a configuration in which the jaws 210, 230 are open. It can be appreciated that, as punch 200 is actuated to move from the open configuration to the closed configuration, complementary cutting edges 222 and 238 bypass each other in a shearing action. In this example, cutting edge 238 is shaped to meet cutting edge 238 at an angle at nearly every position in the actuation cycle, such as for example when the jaws approach the closed position. Shearing thus takes place at a point of contact with the angled cutting edges, as distinguished from a line of contact which would occur with parallel cutting edges. Complementary cutting edges 228, 242 also bypass each other in a shearing action. As punch 200 moves from the open configuration to the closed configuration with a piece of tissue interposed between the jaws, the tissue will be grasped between the jaws 210, 230 and cut or sheared between edges 228, 238 and between edges 228, 242. A single actuation of the punch 200 takes a large bite of tissue and produces two smaller pieces of tissue debris. Furthermore, as the jaws 210, 230 close, the jaw 230 pushes the smaller pieces of tissue through the aperture 216 to eject the tissue from the punch 200. The aperture 216 may be flared or drafted to be larger on the outer side 218 to assist in ejecting debris from the punch 200. Therefore, the jaw 230 may be considered a means for ejecting the debris from the punch.

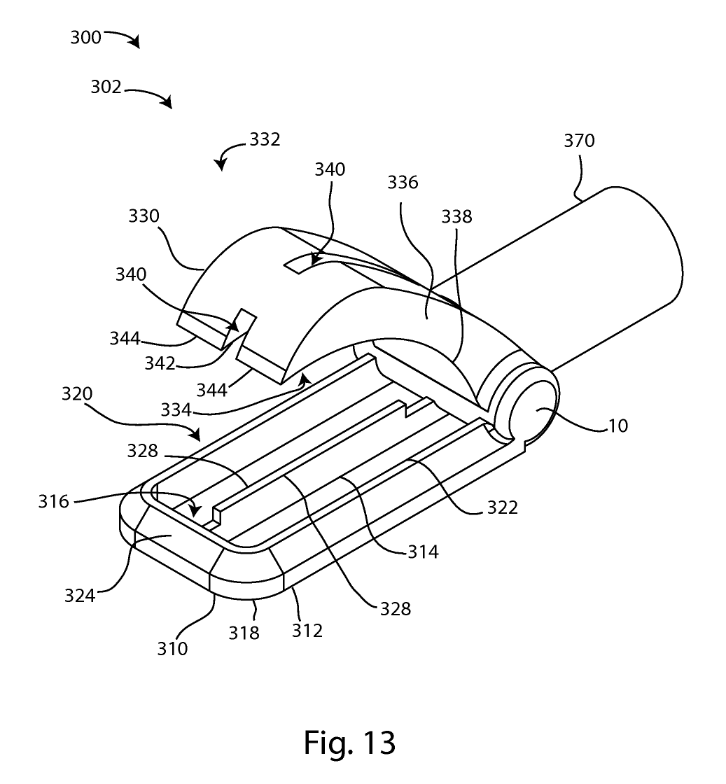

In FIG. 13, a working tip 302 of a punch 300 is illustrated. The remainder of punch 300 may share some or all of the characteristics set forth elsewhere in this description with regard to other punches. The working tip 302 may cut a single bite of tissue into two pieces. Working tip 302 may include a first jaw 310 and a second jaw 330.

The first jaw 310 includes a frame, rim, or loop 312 and a crossbar 314. The loop 312 encircles an aperture 316 which extends through the first jaw 310 between an outer side 318 and an inner side 320 of the first jaw 310. In this example, the aperture 316 is generally rectangular. A cutting edge 322 may extend at least partially around the loop 312 on the inner side 320. For example, the cutting edge 322 may be the edge at the intersection of the aperture 316 and the inner side 320 of the first jaw 310, as illustrated. The first jaw 310 may include one or more bevels 324. For example, bevel 324 may exist on the inner side 320 of the loop 312, and may be complementary to the cutting edge 322. The crossbar 314 spans, or bridges, the aperture 316 generally longitudinally along the first jaw 310. The crossbar 314 may be said to follow a path that intersects the loop 312. The crossbar 314 includes at least one cutting edge 328 on the inner side 320; FIG. 13 illustrates a crossbar with two cutting edges.

The second jaw 330 may have an outer side 332 and an inner side 334 with side walls 336 extending along the second jaw 330 between the outer and inner sides 332, 334. The second jaw 330 may have a profile which is complementary to at least a portion of the aperture 316 of the first jaw 310. In other words, the second jaw 330 may fit the aperture 316, for example with a clearance fit or an interference fit. Referring to FIG. 13, one can appreciate that second jaw 330 and aperture 316 have complementary generally rectangular profiles. The second jaw 330 includes a cutting edge 338 on the inner side 334, which may be, for example, the edge at the intersection of the side wall 336 and the inner side 334. The side wall 336 may intersect the inner side 334 at an acute angle in order to sharpen the cutting edge 338. For example, the side wall may be drafted, relieved, or ground to sharpen the cutting edge 338. The second jaw 330 may include another cutting edge 344 between cutting edges 338. The second jaw 330 includes a recess, slot, channel, or groove 340 which extends along the second jaw 330. In this example, the groove 340 extends through the second jaw 300. The groove 340 includes at least one cutting edge 342 on the inner side 334; FIGS. 6A-6B illustrate a groove with two cutting edges (one is hidden).

The second jaw 330 may be hinged to the first jaw 310 with fastener 10 in a manner similar to that described elsewhere in this description with regard to other punches.

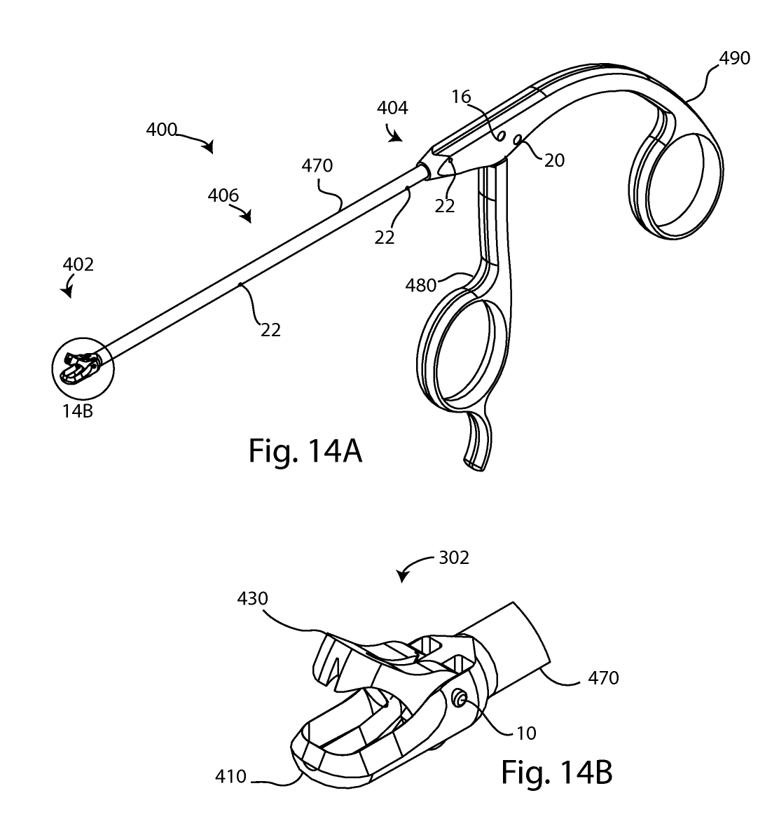

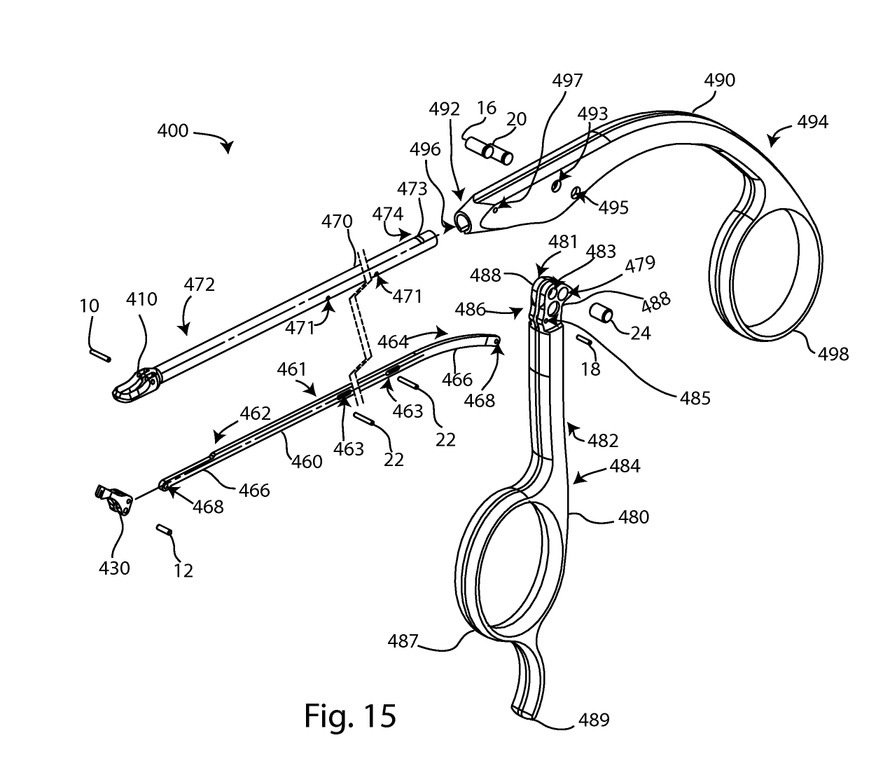

In FIGS. 14A-19B, yet another punch 400 is illustrated. Punch 400 may include first jaw 410, second jaw 430, first shaft 460, second shaft 470, first handle 480, and second handle 490. Punch 400 may be described as having a working tip 402 and a handle portion 404 separated by a shaft portion 406. The working tip 402 includes means for cutting a single bite of tissue and subdividing the bite into multiple pieces. The single bite of tissue may be described as a waste portion of the tissue. The multiple pieces may be described as debris portions of the tissue. Working tip 402 may include the first and second jaws 410, 430, and may also include an adjacent portion of the first and/or second shafts 460, 470. The handle portion 404 includes means for actuating the punch 400 to cut and subdivide the bite of tissue. Handle portion 404 may include the first and second handles 480, 490, and may also include an adjacent portion of the first and/or second shafts 460, 470. The shaft portion 406 includes at least a middle portion of the first and second shafts 460, 470.

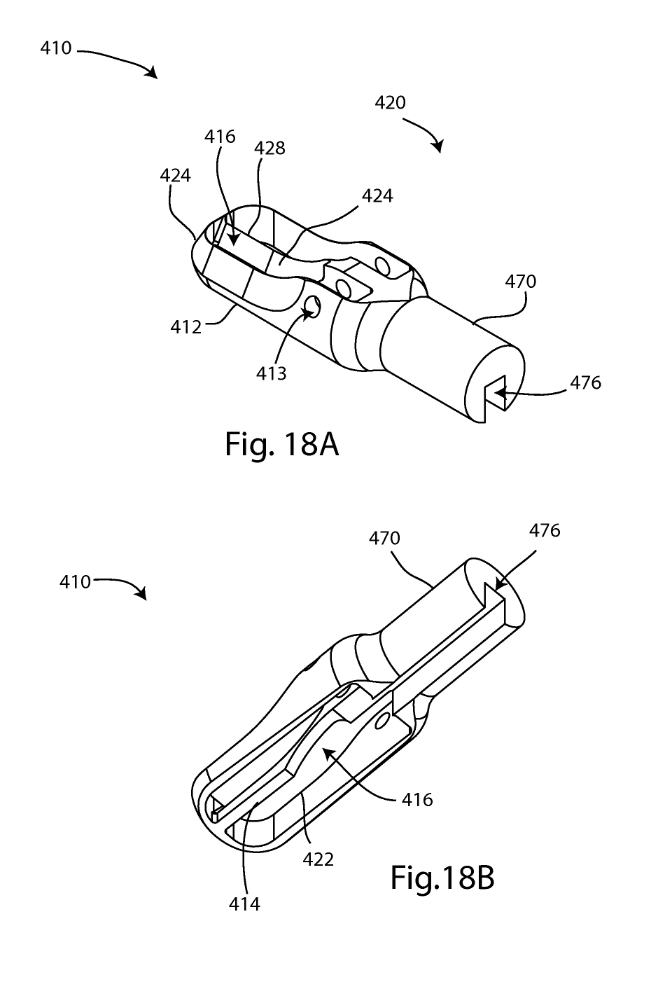

With primary reference to FIGS. 18A-18B, the first jaw 410 includes a frame, rim, or loop 412 and a crossbar 414. The loop 412 encircles an aperture 416 which extends through the first jaw 410 between an outer side 418 and an inner side 420 of the first jaw 410. The aperture 416 may be described as piercing the first jaw 410. The aperture 416 may have a profile that is oblong, rectangular, polygonal, oval, round, or otherwise forms a boundary or perimeter of a simple 2-dimensional region, otherwise known as a closed shape. For example, the aperture 416 illustrated in FIGS. 18A-18B may be described as oblong. A cutting edge 422 may extend at least partially around the loop 412 on the inner side 420. For example, the cutting edge 422 may be the edge at the intersection of the aperture 416 and the inner side 420 of the first jaw 410, as illustrated in FIGS. 18A-18B. The cutting edge 422 may be described as following a majority, or at least a portion, of the profile of the aperture 416. The first jaw 410 may include one or more bevels 424. For example, bevel 424 may exist on the inner side 420 of the loop 412, and may be complementary to the cutting edge 422. The crossbar 414 extends generally longitudinally along the first jaw 410 so as to span, or bridge, the aperture 416. The crossbar 414 may be said to follow a path that intersects the loop 412. The crossbar 414 may be flush with the outer side 418 and recessed below the cutting edge 422. The crossbar 414 includes at least one cutting edge 428 on the inner side 420; FIGS. 18A-18B illustrate a crossbar with one cutting edge and a bevel 424. The first jaw 410 may be formed as a single piece with the second shaft 470, as shown in this example; FIGS. 18A-18B show a truncated portion of second shaft 470. The first jaw 410 may include a transverse through hole 413, which in this example is adjacent to a transition between the first jaw and the second shaft 470. The transverse hole 413 is perpendicular to the crossbar 414 and intersects the aperture 416.

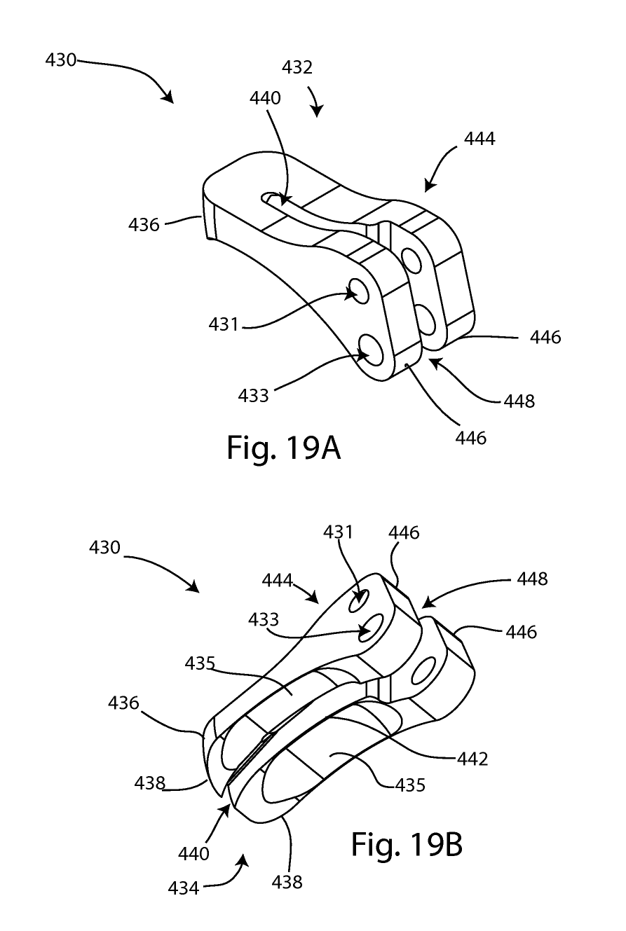

With primary reference to FIGS. 19A-19B, the second jaw 430 may have an outer side 432 and an inner side 434 with a side wall 436 extending around the second jaw 430 between the outer and inner sides 432, 434. The side wall 436 may form a profile which is complementary to at least a portion of the aperture 416 of the first jaw 410. In other words, the side wall 436 may fit the aperture 416, for example with a clearance fit or an interference fit. Referring to FIGS. 14B and 18A-19B, one can appreciate that second jaw 430 and aperture 416 have complementary oblong profiles. The second jaw 430 includes a cutting edge 438 on the inner side 434, which may be, for example, the edge at the intersection of the side wall 436 and the inner side 434. The side wall 436 may intersect the inner side 434 at an acute angle in order to sharpen the cutting edge 438. For example, the side wall 436 may be drafted, relieved, or ground to sharpen the cutting edge 438. Alternatively, the inner side 434 may be drafted, relieved, or ground; second jaw 430 is shown with dished indentations 435 which may also be referred to as relief. The second jaw 430 includes a recess, slot, channel, or groove 440 which extends generally longitudinally along the second jaw 430. In this example, the groove 440 extends through the second jaw 430 between the inner and outer sides 434, 432. The groove 440 includes at least one cutting edge 442 on the inner side 434. The second jaw 430 may form a fork or clevis 444 extending from a narrow end of the oblong profile. The clevis 444 may be described as a plurality of arms 446 separated by at least one slot 448. The clevis 444 may include a transverse through hole 431, which in the illustrated example is proximate the outer side adjacent to terminal ends of the arms 446. Hole 431 is perpendicular to the groove 440. The clevis 444 may also include another transverse through hole 433, which in the illustrated example is proximate the inner side 434 adjacent to free ends of the arms 446.

The second jaw 430 may be hinged to the first jaw 410 with fastener 10 in holes 413, 431 so that the inner sides 420, 434 are facing, the second jaw 430 fits the aperture 416, and the groove 440 receives the crossbar 414. One can appreciate that the second jaw 430 may rotate relative to the first jaw 410 about an axis of rotation of fastener 10, which may be a screw or pin. For example, second jaw 430 may rotate relative to first jaw 410 about a central longitudinal axis of fastener 10. It can be appreciated that the hinged connection between the first and second jaws 410, 430 may serve to close off a reduced portion of the aperture 416. In this sense, the hinged connection may be considered to form a portion of a perimeter of the reduced portion of the aperture 416.

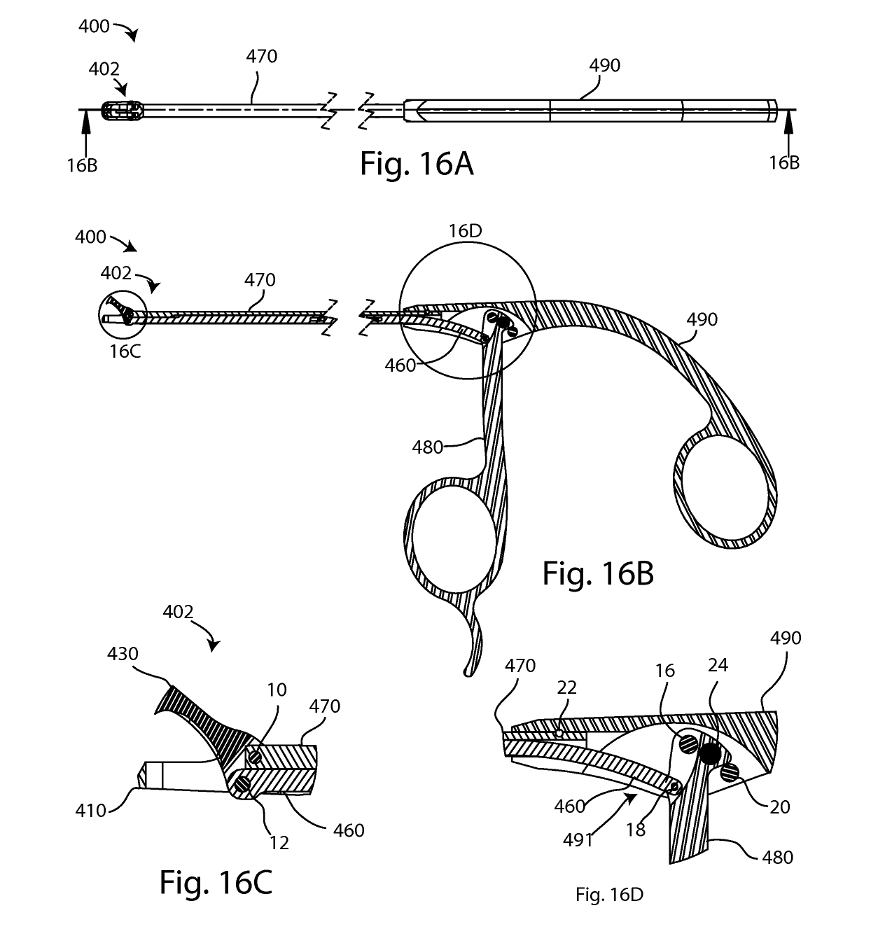

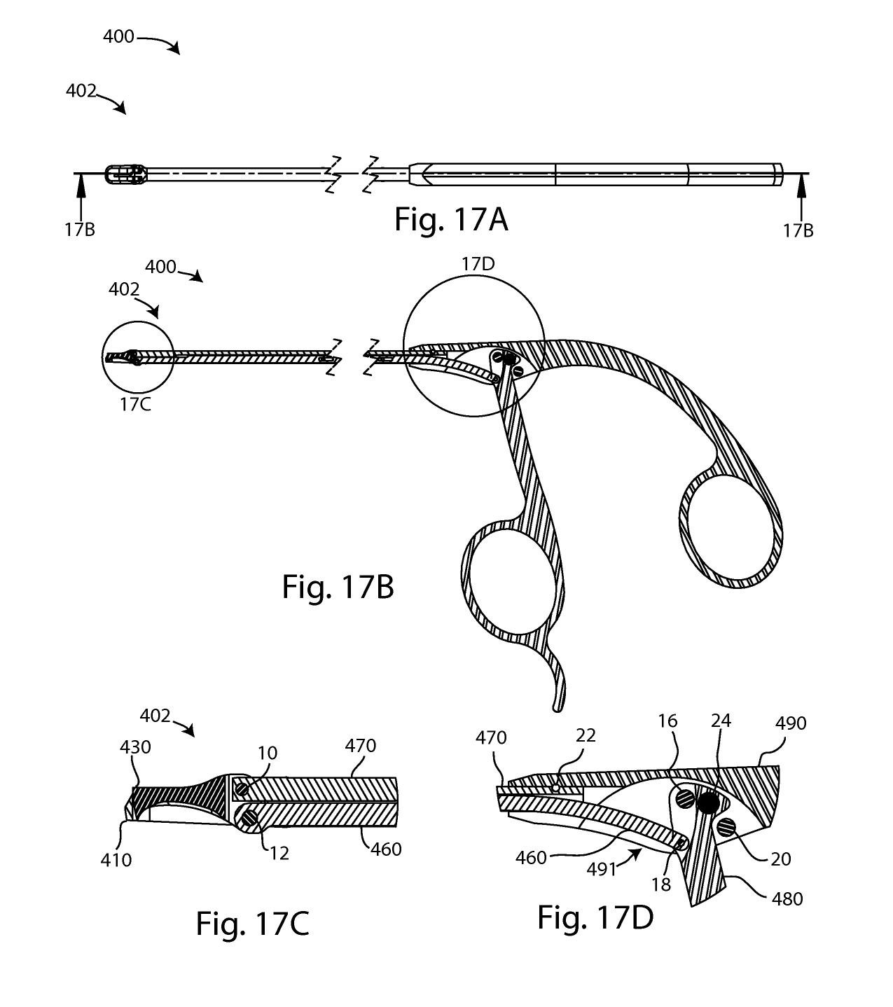

Referring to FIGS. 14A-17D, the first shaft 460 is an elongated member that extends between a first end portion 462 and a second end portion 464. Each end portion 462, 464 may be flattened so as to form a tang 466, and each end portion may include a through hole 468. Each end portion 462, 464 may be positioned eccentrically relative to a mid portion 461 of the first shaft 460. For example, the tang 466 of first end portion 462 is eccentrically laterally enlarged (FIGS. 16C and 17C), while second end portion 464 is bent (FIGS. 16D and 17D). First shaft 460 may include one or more slots 463 distributed along its length. First shaft 460 may be rigid or flexible, solid or segmented. For example, first shaft 460 may be a multi-stranded cable. In this example, first shaft 460 is shown with a rectangular cross section.

The first shaft 460 may be hinged to the second jaw 430 with fastener 12 in holes 433, 468 so that the tang 466 on the first end portion 462 fits in the slot 448 of the second jaw.

The second shaft 470 is an elongated member that extends between a first end portion 472 and a second end portion 474. In this example, the second shaft 470 is formed as a single piece with the first jaw 410. The second shaft 470 is a solid member with a longitudinal slot 476, best seen in FIGS. 18A-18B. The second shaft 470 may include one or more holes 471 distributed along its length. The second end portion 474 may include a groove or notch 473.

The second shaft 470 may slidingly receive the first shaft 460 within the slot 476. The first and second shafts 460, 470 may be secured together by fasteners 22 in holes 471 and slots 463.

The first handle 480 is an elongated member that extends between a first end portion 482 and a second end portion 484. The first handle 480 may be described as a movable handle. The first end portion 482 may terminate in a bifurcation or clevis 486. The clevis 486 may be described as a plurality of arms 488 separated by at least one slot 481. The clevis 486 may include a transverse through hole 483, which in the illustrated example is proximate terminal ends of the arms 488. The clevis 486 may also include another transverse through hole 485, which in the illustrated example is at an intermediate location along the arms 488. The clevis 486 may include yet another transverse through hole 479 next to hole 483. The second end portion 484 may include a loop 487 and/or a rest 489. A user may insert one or more digits through the loop 487 or may rest one or more digits on the rest 489.

The first handle 480 may be hinged to the first shaft 460 with fastener 18 in holes 485, 468 so that the tang 466 on the second end portion 464 fits in the slot 481 of the first handle. Fastener 24 may be secured in hole 479. Fastener 24 may, for example, function as an attachment point for a spring (not shown) or as a drag plug.

The second handle 490 is an elongated member that extends between a first end portion 492 and a second end portion 494. The second handle 490 may be described as a stationary handle. The first end portion 492 may terminate in a central longitudinal hole 496. A transverse through hole 497 may extend through the first end portion 492 more or less tangent to the hole 496. The second end portion 494 may include a loop 498, which may resemble loop 487 in appearance and/or function. The second handle 490 may include an indentation, recess, or alcove 491 at an intermediate location between the first and second end portions 492, 494, as may be seen best in FIGS. 16D and 17D. The second handle 490 may include a transverse through hole 493 which intersects, or crosses, the alcove 491. The second handle 490 may include another transverse through hole 495 which also crosses the alcove 491. The holes 493, 495 may be on opposite sides of the hole 496, as shown.

The second handle 490 may be hinged to the first handle 480 with fastener 16 in holes 493, 483 so that alcove 491 receives clevis 486 and hole 485 is proximate hole 495 (FIGS. 16D and 17D). Fastener 20 may be secured in hole 495 to limit the rotation of first handle 480 relative to second handle 490. Hole 496 may receive the second end portion 474 of second shaft 470. The second handle 490 and the second shaft 470 may be permanently or releasably connected in a manner similar to that set forth elsewhere in this description with regard to other second handles and second shafts. In FIGS. 16D and 17D, the second handle 490 and the second shaft 470 are connected by inserting fastener 22 in hole 497 and groove 473.

When punch 400 is operatively assembled, the second handle 490, second shaft 470, and first jaw 410 are firmly fixed together and the first handle 480, first shaft 460, and second jaw 430 are linked together so that rotation of first handle 480 relative to second handle 490 about fastener 16 causes corresponding rotation of second jaw 430 relative to first jaw 410 about fastener 10. The illustrated example of punch 400 is configured so that when first handle 480 abuts fastener 20, second jaw 430 fits at least partially within aperture 416 of first jaw 410, as may be seen best in FIGS. 17A-17D. In other words, FIGS. 17A-17D show punch 400 in a configuration in which the jaws 410, 430 are closed. When first handle 480 is rotated away from fastener 20, second jaw 430 tilts away from first jaw 410, as may be seen best in FIGS. 16A-16D. In other words, FIGS. 16A-16D show punch 400 in a configuration in which the jaws 410, 430 are open. It can be appreciated that, as punch 400 is actuated to move from the open configuration to the closed configuration, complementary cutting edges 422 and 438 bypass each other in a shearing action. Similarly, complementary cutting edges 428, 442 bypass each other in a shearing action. As punch 400 moves from the open configuration to the closed configuration with a piece of tissue interposed between the jaws, the tissue will be grasped between the jaws 410, 430 and cut or sheared between edges 428, 438 and between edges 428, 442. A single actuation of the punch 400 takes a large bite of tissue and produces two smaller pieces of tissue debris. Furthermore, as the jaws 410, 430 close, the jaw 430 pushes the smaller pieces of tissue through the aperture 416 to eject the tissue from the punch 400. The aperture 416 may be flared or drafted to be larger on the outer side 418 to assist in ejecting debris from the punch 400. Therefore, the jaw 430 may be considered a means for ejecting the debris from the punch.

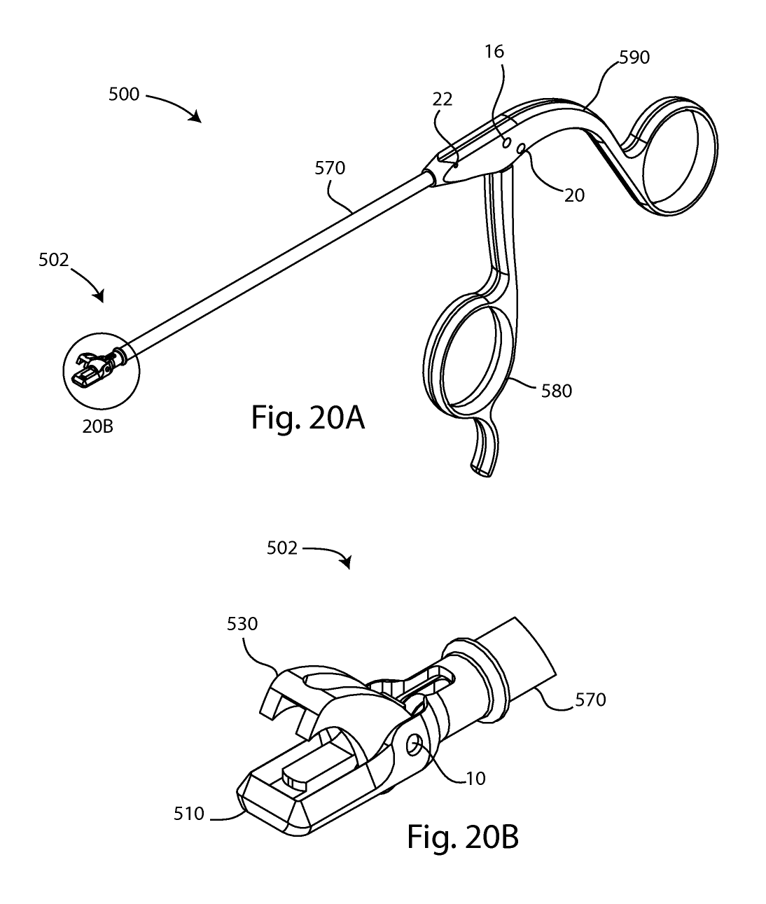

In FIGS. 20A-25B, yet another punch 500 is illustrated. Punch 500 may include first jaw 510, second jaw 530, link 550, first shaft 560, second shaft 570, first handle 580, and second handle 590. Punch 500 may be described as having a working tip 502 and a handle portion 504 separated by a shaft portion 506. The working tip 502 includes means for cutting a single bite of tissue and subdividing the bite into multiple pieces. The single bite of tissue may be described as a waste portion of the tissue. The multiple pieces may be described as debris portions of the tissue. Working tip 502 may include the first and second jaws 510, 530, and may also include an adjacent portion of the first and/or second shafts 560, 570. The handle portion 504 includes means for actuating the punch 500 to cut and subdivide the bite of tissue. Handle portion 504 may include the first and second handles 580, 590, and may also include an adjacent portion of the first and/or second shafts 560, 570. The shaft portion 506 includes at least a middle portion of the first and second shafts 560, 570.

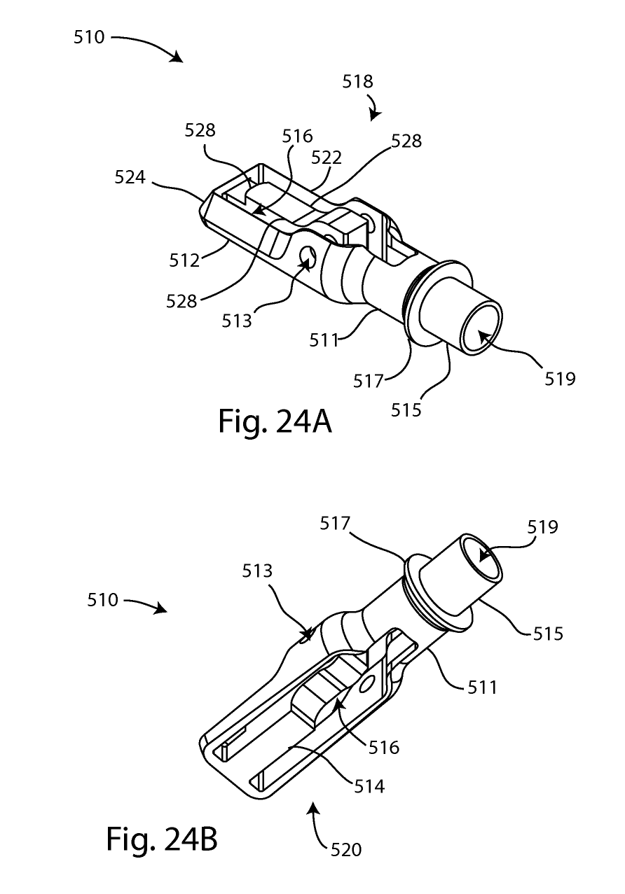

With primary reference to FIGS. 24A-24B, the first jaw 510 includes a frame, rim, or loop 512 and a crossbar 514. The loop 512 encircles an aperture 516 which extends through the first jaw 510 between an outer side 518 and an inner side 520 of the first jaw 510. The aperture 516 may be described as piercing the first jaw 510. The aperture 516 may have a profile that is oblong, rectangular, polygonal, oval, round, or otherwise forms a boundary or perimeter of a simple 2-dimensional region, otherwise known as a closed shape. For example, the aperture 516 illustrated in FIGS. 24A-24B may be described as spoon-shaped or pear-shaped. A cutting edge 522 may extend at least partially around the loop 512 on the inner side 520. For example, the cutting edge 522 may be the edge at the intersection of the aperture 516 and the inner side 520 of the first jaw 510, as illustrated in FIGS. 24A-24B. The cutting edge 522 may be described as following a majority, or at least a portion, of the profile of the aperture 516. The first jaw 510 may include one or more bevels 524. For example, bevel 524 may exist on the inner side 520 of the loop 512, and may be complementary to the cutting edge 522. The crossbar 514 extends generally longitudinally along the first jaw 510 so as to span, or bridge, the aperture 516. The crossbar 514 may be said to follow a path that intersects the loop 512. The crossbar 514 may be flush with the outer side 518 and recessed below the cutting edge 522. In this example, the crossbar 514 may be described as a cantilever beam projecting within the aperture 516. The crossbar 514 includes at least one cutting edge 528 on the inner side 520; FIGS. 24A-24B illustrate a crossbar with three cutting edges. The first jaw 510 may also include a shaft 511 extending from the loop 512. For example, FIGS. 24A-24B illustrate shaft 511 extending from the loop 512 at a narrow end of the spoon-shaped or pear-shaped aperture 516. The shaft 511 may include a transverse through hole 513, which in the illustrated example is adjacent to a transition between the narrow end and a wide end of the spoon-shaped or pear-shaped aperture 516. The transverse hole 513 is perpendicular to the crossbar 514 and intersects the free end of the crossbar 514 and the aperture 516. The shaft 511 may terminate in a boss 515, which may be of a different diameter than the shaft 511. A flange 517 may separate the shaft 511 and the boss 515. The shaft 511 may include a central longitudinal hole 519 which extends between the aperture 516 and the boss 515.

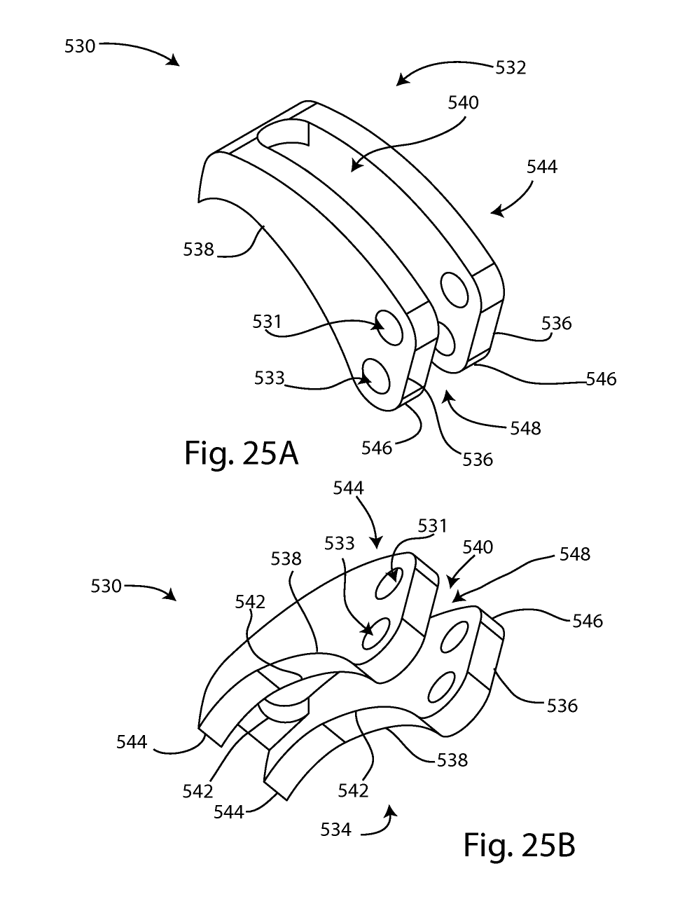

With primary reference to FIGS. 25A-25B, the second jaw 530 may have an outer side 532 and an inner side 534 with side walls 536 extending along the second jaw 530 between the outer and inner sides 532, 534. The second jaw 530 may have a profile which is complementary to at least a portion of the aperture 516 of the first jaw 510. In other words, the second jaw 530 may fit the aperture 516, for example with a clearance fit or an interference fit. Referring to FIGS. 20B and 24A-25B, one can appreciate that second jaw 530 and aperture 516 have complementary profiles, at least in a wide portion of the spoon-shaped or pear-shaped aperture 516. The second jaw 530 includes a cutting edge 538 on the inner side 534, which may be, for example, the edge at the intersection of the side wall 536 and the inner side 534. The side wall 536 may intersect the inner side 534 at an acute angle in order to sharpen the cutting edge 538. For example, the side wall may be drafted, relieved, or ground to sharpen the cutting edge 538. The second jaw 530 may include another cutting edge 544 between edges 538. The second jaw 530 includes a recess, slot, channel, or groove 540 which extends generally longitudinally along the second jaw 530. The groove 540 includes at least one cutting edge 542 on the inner side 534; FIGS. 25A-25B illustrate a groove with three cutting edges. The second jaw 530 may form a fork or clevis 544 extending from a narrow end of the profile. The clevis 544 may be described as a plurality of arms 546 separated by at least one slot 548. The clevis 544 may include a transverse through hole 531, which in the illustrated example is proximate the outer side 532 adjacent to terminal ends of the arms 546. Hole 531 is perpendicular to the groove 540. The clevis 544 may also include another transverse through hole 533, which in the illustrated example is proximate the inner side 534 adjacent to free ends of the arms 546.

The second jaw 530 may be hinged to the first jaw 510 with fastener 10 in holes 513, 531 so that the inner sides 520, 534 are facing, the second jaw 530 fits the aperture 516, and the groove 540 receives the crossbar 514. One can appreciate that the second jaw 530 may rotate relative to the first jaw 510 about an axis of rotation of fastener 10, which may be a screw or pin. For example, second jaw 530 may rotate relative to first jaw 510 about a central longitudinal axis of fastener 10. It can be appreciated that the hinged connection between the first and second jaws 510, 530 may serve to close off a reduced portion of the aperture 516. In this sense, the hinged connection may be considered to form a portion of a perimeter of the reduced portion of the aperture 516.

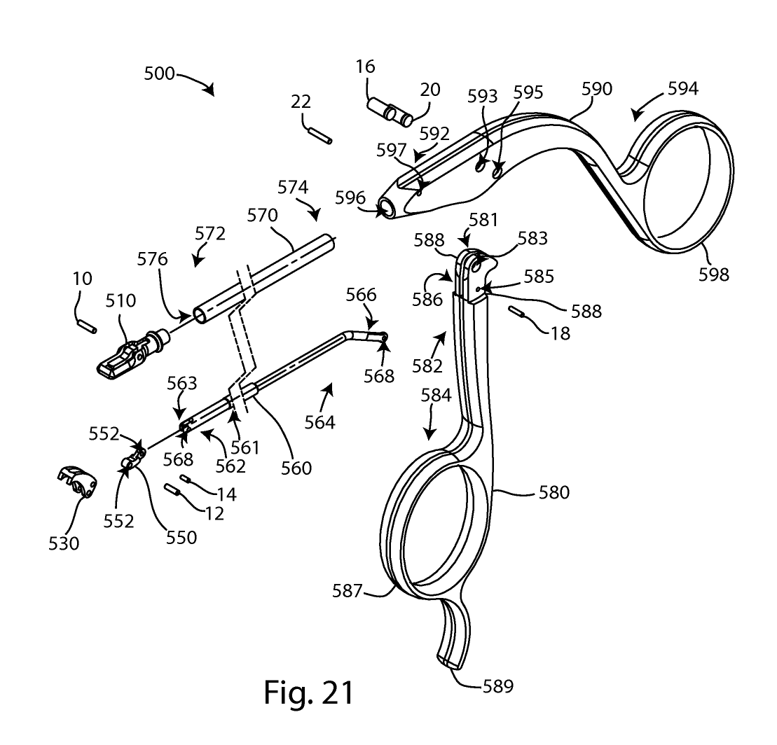

Referring to FIGS. 21-23D, the link 550 may be a kidney-shaped or curved elongated member with transverse through holes 552 at either end. The link 550 may be hinged to the second jaw 530 with fastener 12 in holes 533, 552 so that a portion of the link fits in the slot 548 of the second jaw.

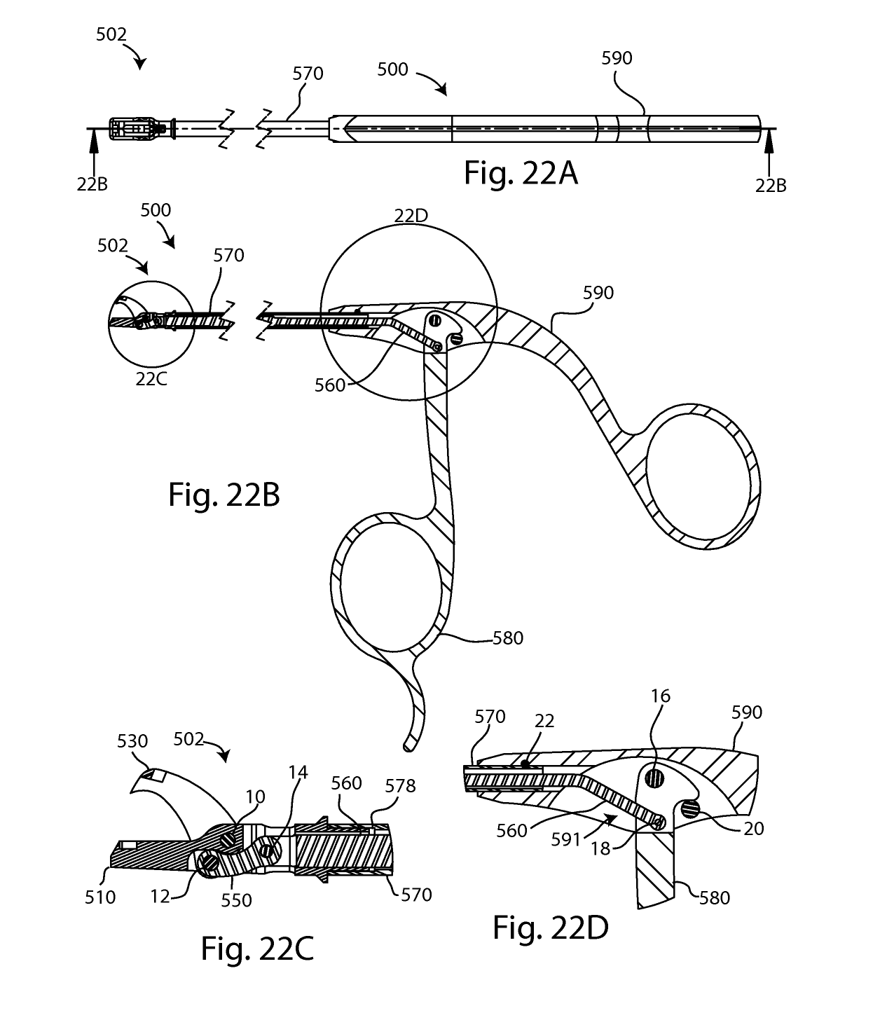

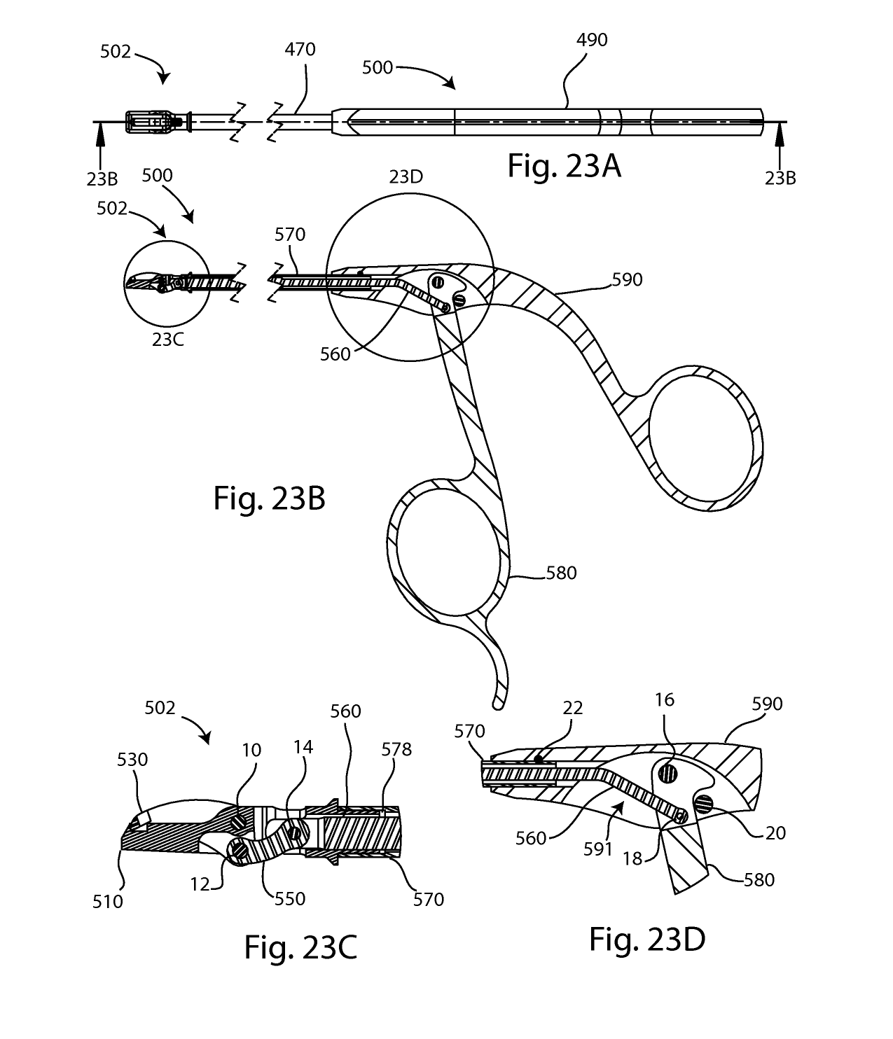

Referring to FIGS. 21-23D, the first shaft 560 is an elongated member that extends between a first end portion 562 and a second end portion 564. At least one end portion 562, 564 may be flattened so as to form a tang 566. Alternately, at least one end portion 562, 564 may form a fork or clevis 563. At least one end portion may include a through hole 568. Each end portion 562, 564 may be positioned eccentrically relative to a mid portion 561 of the first shaft 560. For example, second end portion 564 is bent (FIGS. 22D and 23D). First shaft 560 may be rigid or flexible, solid or segmented. For example, first shaft 560 may be a multi-stranded cable.

The first shaft 560 may be hinged to the link 550 with fastener 14 in holes 568, 552 so that at least a portion of the link 550 fits in the clevis 563 of the first shaft.

The second shaft 570 is an elongated member that extends between a first end portion 572 and a second end portion 574. The illustrated shaft 570 is a tube with a central longitudinal through hole 576. The first end portion 572 may include a socket 578 which may be of a different diameter than the hole 576. For example, the socket 578 may be a counterbore.

The second shaft 570 may slidingly receive the first shaft 560 within the hole 576, and may receive the boss 515 of the first jaw 510 within the socket 578. The second shaft 570 and the first jaw 510 may be permanently connected, such as by welding, soldering, brazing, or gluing, among other processes. The flange 517 may provide filler material for a thermal fastening operation. The second shaft 570 and the first jaw 510 may be releasably connected, such as by threads, snap fit, retaining ring, or interference fit, among other connections.

The first handle 580 is an elongated member that extends between a first end portion 582 and a second end portion 584. The first handle 580 may be described as a movable handle. The first end portion 582 may terminate in a bifurcation or clevis 586. The clevis 586 may be described as a plurality of arms 588 separated by at least one slot 581. The clevis 586 may include a transverse through hole 583, which in the illustrated example is proximate terminal ends of the arms 588. The clevis 586 may also include another transverse through hole 585, which in the illustrated example is at an intermediate location along the arms 588. The second end portion 584 may include a loop 587 and/or a rest 589. A user may insert one or more digits through the loop 587 or may rest one or more digits on the rest 589.

The first handle 580 may be hinged to the first shaft 560 with fastener 18 in holes 585, 568 so that the tang 566 on the second end portion 564 fits in the slot 581 of the first handle.

The second handle 590 is an elongated member that extends between a first end portion 592 and a second end portion 594. The second handle 590 may be described as a stationary handle. The first end portion 592 may terminate in a central longitudinal hole 596. A transverse through hole 597 may extend through the first end portion 592 more or less tangent to the hole 596. The second end portion 594 may include a loop 598, which may resemble loop 587 in appearance and/or function. The second handle 590 may include an indentation, recess, or alcove 591 at an intermediate location between the first and second end portions 592, 594, as may be seen best in FIGS. 22D and 23D. The second handle 590 may include a transverse through hole 593 which intersects, or crosses, the alcove 591. The second handle 590 may include another transverse through hole 595 which also crosses the alcove 591. The holes 593, 595 may be on opposite sides of the hole 596, as shown.

The second handle 590 may be hinged to the first handle 580 with fastener 16 in holes 593, 583 so that alcove 591 receives clevis 586 and hole 585 is proximate hole 595 (FIGS. 22D and 23D). Fastener 20 may be secured in hole 595 to limit the rotation of first handle 580 relative to second handle 590. Hole 596 may receive the second end portion 574 of second shaft 570. The second handle 590 and the second shaft 570 may be permanently or releasably connected in a manner similar to that set forth above with regard to second shaft 570 and first jaw 510. If needed, sacrificial filler material may be provided on the second handle 590 or second shaft 570.

When punch 500 is operatively assembled, the second handle 590, second shaft 570, and first jaw 510 are firmly fixed together and the first handle 580, first shaft 560, and second jaw 530 are linked together so that rotation of first handle 580 relative to second handle 590 about fastener 16 causes corresponding rotation of second jaw 530 relative to first jaw 510 about fastener 10. The illustrated example of punch 500 is configured so that when first handle 580 abuts fastener 20, second jaw 530 fits at least partially within aperture 516 of first jaw 510, as may be seen best in FIGS. 23A-23D. In other words, FIGS. 23A-23D show punch 500 in a configuration in which the jaws 510, 530 are closed. When first handle 580 is rotated away from fastener 20, second jaw 530 tilts away from first jaw 510, as may be seen best in FIGS. 22A-22D. In other words, FIGS. 22A-22D show punch 500 in a configuration in which the jaws 510, 530 are open. It can be appreciated that, as punch 500 is actuated to move from the open configuration to the closed configuration, complementary cutting edges 522 and 538 bypass each other in a shearing action. Similarly, complementary cutting edges 528, 542 bypass each other in a shearing action. As punch 500 moves from the open configuration to the closed configuration with a piece of tissue interposed between the jaws, the tissue will be grasped by the jaws 510, 530 and cut or sheared between edges 528, 538 and between edges 528, 542. A single actuation of the punch 500 takes a large bite of tissue and produces three smaller pieces of tissue debris. Furthermore, as the jaws 510, 530 close, the jaw 530 pushes the smaller pieces of tissue through the aperture 516 to eject the tissue from the punch 500. The aperture 516 may be flared or drafted to be larger on the outer side 518 to assist in ejecting debris from the punch 500. Therefore, the jaw 530 may be considered a means for ejecting the debris from the punch.

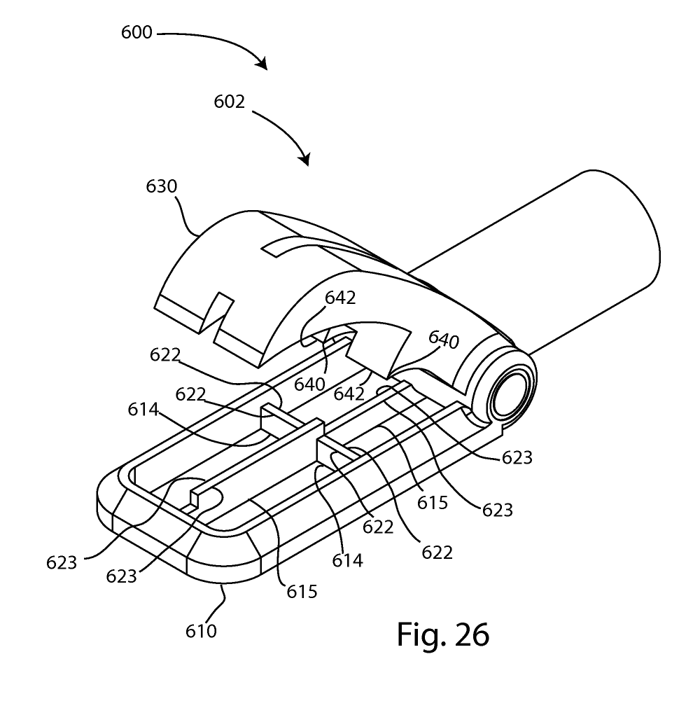

In FIG. 26, a working tip 602 of a punch 600 is illustrated. The remainder of punch 600 may share some or all of the characteristics set forth elsewhere in this description with regard to other punches. The working tip 602 includes means for cutting a single bite of tissue into four pieces. Working tip 602 may include a first jaw 610 and a second jaw 630.

The first jaw 610 includes a first crossbar 614 and a second crossbar 315. The remainder of the first jaw 610 may share some or all of the characteristics set forth elsewhere in this description with regard to other first jaws, such as those set forth for first jaw 310. The crossbar 614 extends transversely across the first jaw 610 and the crossbar 615 extends generally longitudinally along the first jaw 610. The crossbars 614, 615 include cutting edges 622, 623 as shown in FIG. 26.

The second jaw 630 includes a crossbar 640. The remainder of the second jaw 630 may share some or all of the characteristics set forth elsewhere in this description with regard to other second jaws, such as those set forth for second jaw 330. The crossbar 640 extends transversely across the second jaw 630. The crossbar 640 includes at least one cutting edge 642, which may be interrupted, as shown in FIG. 26.

The second jaw 630 may be hinged to the first jaw 610 with fastener 10 in a manner similar to that described elsewhere in this description with regard to other punches.

It should be understood that the present components, systems, kits, apparatuses, and methods are not intended to be limited to the particular forms disclosed. Rather, they are intended to include all modifications, equivalents, and alternatives falling within the scope of the claims. They are further intended to include embodiments which may be formed by combining features from the disclosed embodiments, and variants thereof.

Certain elements disclosed herein may be interpreted as means for grasping a waste portion of a tissue. For example, the complementary first and second jaws disclosed herein may grasp a waste portion of a tissue. In another example, the facing inner sides of the jaws disclosed herein may include tissue grasping adaptations.

Certain elements disclosed herein may be interpreted as means for cutting a waste portion of a tissue from a main portion of the tissue. For example, the complementary peripheral cutting edges disclosed herein may cooperate to cut a waste portion of a tissue.

Certain elements disclosed herein may be interpreted as means for subdividing a waste portion of a tissue into a plurality of debris portions. For example, the complementary crossbar cutting edges disclosed herein may cooperate to subdivide a waste portion of a tissue.

Certain elements disclosed herein may be interpreted as means for actuating an instrument to grasp, cut, subdivide, and/or eject the waste portion. For example, the linkages disclosed herein (which may include a first handle, first shaft, optional link, and second jaw) may actuate an instrument by transmitting and/or transforming a force input into an instrument action.

Certain elements disclosed herein may be interpreted as means for ejecting debris portions of a tissue from an instrument. For example, the complementary first and second jaws disclosed herein may eject debris.

The claims are not to be interpreted as including means-plus- or step-plus-function limitations, unless such a limitation is explicitly recited in a given claim using the phrase(s) “means for” or “step for,” respectively.

The term “coupled” is defined as connected, although not necessarily directly, and not necessarily mechanically.

The use of the word “a” or “an” when used in conjunction with the term “comprising” in the claims and/or the specification may mean “one,” but it is also consistent with the meaning of “one or more” or “at least one.” The term “about” means, in general, the stated value plus or minus 5%. The use of the term “or” in the claims is used to mean “and/or” unless explicitly indicated to refer to alternatives only or the alternative are mutually exclusive, although the disclosure supports a definition that refers to only alternatives and “and/or.”

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements, possesses those one or more steps or elements, but is not limited to possessing only those one or more elements. Likewise, a step of a method or an element of a device that “comprises,” “has,” “includes” or “contains” one or more features, possesses those one or more features, but is not limited to possessing only those one or more features. Furthermore, a device or structure that is configured in a certain way is configured in at least that way, but may also be configured in ways that are not listed.

1. An instrument, comprising:
a first jaw, wherein the first jaw comprises an aperture at least partially encircled by a first cutting edge;
a second jaw connected to the first jaw by a hinge, wherein the second jaw rotates relative to the first jaw about an axis of rotation of the hinge; and
a second cutting edge, wherein the second cutting edge spans the aperture.

2. The instrument of claim 1, wherein the second cutting edge extends parallel to the axis.

3. The instrument of claim 1, wherein the second cutting edge extends perpendicular to the axis.

4. The instrument of claim 1, wherein the second cutting edge is carried by the first jaw.

5. The instrument of claim 1, wherein the second cutting edge is carried by the second jaw.

6. The instrument of claim 1, comprising:
a third cutting edge, wherein the third cutting edge spans the aperture.

7. The instrument of claim 6, wherein the third cutting edge crosses the second cutting edge.

8. A surgical punch, comprising:
a first cutting edge, wherein the first cutting edge extends along a majority of a perimeter around a simple two-dimensional region; and
a second cutting edge, wherein the second cutting edge crosses the region, wherein the second cutting edge extends along a path that intersects the first cutting edge.

9. The punch of claim 8, wherein the punch comprises:
a pair of opposing jaws, wherein the first and second cutting edges are carried by the jaws.

10. The punch of claim 9, wherein the jaws are connected by a hinge, wherein the hinge extends along a remainder of the perimeter.

11. The punch of claim 10, wherein the second cutting edge is parallel to the hinge.

12. The punch of claim 10, wherein the second cutting edge is perpendicular to the hinge.

13. The punch of claim 8, wherein the punch comprises:
a third cutting edge, wherein the third cutting edge crosses the region, wherein the third cutting edge extends along a path that intersects the first cutting edge.

14. The punch of claim 13, wherein the third cutting edge extends along a path that intersects the second cutting edge.

15. A surgical punch, comprising:
means for cutting a waste portion of a tissue from a main portion of the tissue;
means for subdividing the waste portion into a plurality of debris portions; and
means for actuating the punch to cut and subdivide the waste portion;
wherein a single actuation of the punch actuates the means for cutting and subdividing the waste portion.

16. The punch of claim 15, wherein the punch comprises:
a hinged pair of opposing jaws, wherein the means for cutting and subdividing the waste portion are carried on the jaws.

17. The punch of claim 15, wherein the punch comprises:
means for grasping the waste portion.

18. The punch of claim 17, wherein a single actuation of the punch actuates the means for grasping, cutting, and subdividing the waste portion.

19. The punch of claim 15, wherein the punch comprises:
means for ejecting the debris portions.

20. The punch of claim 19, wherein a single actuation of the punch actuates the means for cutting the waste portion, the means for subdividing the waste portion, and the means for ejecting the debris portions.
